(19) United States
(12) Patent Application Publication
(10) Pub. No.: US 2025/0195877 A1
(43) Pub. Date: Jun. 19, 2025

(54) PERSONALIZED THERAPY NEUROSTIMULATION SYSTEMS

(71) Applicant: Cala Health, Inc., San Mateo, CA (US)

(72) Inventors: Gregory T. Schulte, Oakland, CA (US); Shengzhi Li, Mountain View, CA (US); Alexander R. Kent, Mountain View, CA (US)

(21) Appl. No.: 18/576,237

(22) PCT Filed: Jul. 6, 2022
(86) PCT No.: PCT/US2022/073451

§ 371 Date: Jan. 3, 2024

Related U.S. Application Data
(60) Provisional application No. 63/203,155, filed on Jul. 9, 2021.

(51) Int. Cl. A61N 1/04; A61B 5/00; A61N 1/36; G16H 20/30

(52) U.S. Cl. CPC A61N 1/0484; A61B 5/4806; A61B 5/4836; A61B 5/7264; A61N 1/0456; A61N 1/36025; A61N 1/36031; G16H 20/30

(57) ABSTRACT

Systems, devices, and methods for electrically stimulating peripheral nerve(s) to treat various disorders are disclosed, as well as signal processing systems and methods for enhancing diagnostic and therapeutic protocols relating to the same. Personalized therapy based on algorithms and aggregated data are also provided.

### REFERENCE TO RELATED APPLICATION

This application claims the benefit of U.S. Provisional Application No. 63/203,155, filed Jul. 9, 2021, the entire disclosure of which is hereby incorporated by reference in its entirety.

### TECHNICAL FIELD

Some embodiments of the invention relate generally to systems, devices, and methods for neuromodulating (such as stimulating) nerves, and more specifically relate to system, devices, and methods for electrically stimulating peripheral nerve(s) to treat disorders and/or associated symptoms, as well as signal processing systems and methods employing reinforcement learning and/or machine learning for personalizing therapeutic protocols relating to the same.

### BACKGROUND

A wide variety of modalities can be utilized to neuromodulate peripheral nerves. For example, Applicant's own work has demonstrated that electrical energy can be delivered transcutaneous via electrodes on the skin surface with neurostimulation systems to stimulate peripheral nerves, including on a patient's limb.

### SUMMARY

Systems (such as partially or fully wearable systems) to neuromodulate nerves with compact, ergonomic form factors are needed to enhance efficacy, compliance, and/or comfort with using the devices. Several embodiments of the system disclosed herein employ machine learning algorithms to optimize therapy parameters based on individual response/satisfaction and assign a therapy response profile to influence further therapy titration. Several embodiments of the system can add additional parameters and parameter options to the therapy profiles over time. Additional data sources may strengthen the machine learning. For example, data can be collected from multiple patients, including data/feedback aggregated from subjective patient feedback and objective patient-specific data from third party devices. The information can be collected, shared, analyzed over the cloud or communicated directly to the wearable system.

In some embodiments, the aggregated data is taken from subpopulations of patients with like characteristics to the treated patient. In some embodiments, the subpopulations may be groups of patients with similar characteristics, e.g., age ranges, gender, hormone levels, drugs used, and/or tremor characteristics.

In some embodiment, the system (such as a partially or fully wearable system) collects kinematic data passively at various intervals throughout daily use. The collection of passive data may be used to determine how therapy impacts response duration and overall satisfaction over time. In this way, the patient is not burdened with inputting satisfaction data into the wearable system. In certain embodiments, a correlation between collected kinematic data and patient satisfaction is leveraged so that future collected kinematic data can be used along with the correlation and/or machine learning to predict patient satisfaction with tremor level and tremor improvements during activity.

In some embodiments, disclosed herein is a neurostimulation device (such as a band, strap, bracelet, cuff or other device that partially or fully encircles a limb) for transcutaneously stimulating one or more peripheral nerves of a user. In some embodiments, the device includes (for example, comprises, consists, or consists essentially of) one or more electrodes configured to generate electric stimulation signals based on therapy parameters. The therapy parameters can be selected from a plurality of predefined profiles determined by an aggregation learning algorithm. The aggregation learning algorithm predicts a plurality of outcomes for the user based on a plurality of predefined profiles. The predefined profiles being based on features extracted from data for a plurality of users. The device can further include one or more sensors configured to detect motion signals, wherein the one or more sensors are operably connected to the neurostimulation device. The device can further include one or more hardware processors configured to perform a therapy session with the therapy parameters, measure kinematic data including data from the detected motion signals during the therapy session, wherein the data optionally includes tremor data, adjust the therapy parameters based on measured data from the detected motion signals during the therapy session, and optimize the adjusted therapy parameters by a learning algorithm based on an individual database. The learning algorithm predicts a modified outcome based on the individual database which includes an aggregation of kinematic data and satisfaction data from multiple therapy sessions for the user. In some embodiments, the individual database includes kinematic data accumulated over multiple hours/days/weeks/months and/or satisfaction data accumulated over multiple hours/days/weeks/months. The device may be partially or fully wearable systems.

In some embodiments, disclosed herein is a neurostimulation device for stimulating one or more peripheral nerves of a user. In some embodiments, the device includes (for example, comprises, consists, or consists essentially of) one or more electrodes configured to generate electric stimulation signals based on therapy parameters, one or more sensors configured to detect motion signals, wherein the one or more sensors are operably connected to the neurostimulation device, and one or more hardware processors configured to perform a therapy session with the therapy parameters, collect passive kinematic data throughout the day of the therapy session, the passive kinematic data being indicative of activities performed by the user at times outside of the therapy session, compare the passive kinematic data to a predicted user satisfaction database, the predicted user satisfaction database including an aggregation of passive kinematic data from multiple therapy sessions, and determine a user satisfaction level for the therapy session based on the comparison. Optionally, the device is a wearable transcutaneous device.

In some embodiments, the features extracted from the data include test kinematic or motion data, satisfaction data or other objective/subjective data. Kinematic data can be collected, for example, from one or more sensors (e.g., onboard the neurostimulation device) or via an input system. Kinematic data can include, as an example, raw accelerometer and/or gyroscope data.

In some embodiments, the therapy parameters are based at least in part on a predetermined decision tree and/or one or more user profiles. In some embodiments, the features extracted from the data for the plurality of users include one or more of geospatial data, temporal data, disease, patient attributes or characteristics, or neurostimulation device characteristics associated with the extracted features.

In some embodiments, one or more sensors are further configured to collect test kinematic data during the therapy session, compare the test kinematic data to a predicted user test satisfaction database, the predicted user test satisfaction database including an aggregation of test kinematic data from multiple therapy sessions for the user, and determine a user test satisfaction level for the therapy session based on the comparison.

In some embodiments, the one or more sensors are further configured to employ an aggregation learning algorithm to determine the user satisfaction level and/or a learning algorithm to determine the user satisfaction level.

In some embodiments, disclosed herein is a neurostimulation device for stimulating one or more peripheral nerves of a user. In some embodiments the device includes (for example, comprises, consists, or consists essentially of) one or more electrodes configured to generate electric stimulation signals based on therapy parameters, one or more sensors configured to detect motion signals, wherein the one or more sensors are operably connected to the neurostimulation device, and one or more hardware processors configured to receive sets of therapy parameters for a trial period, select a first set from the sets of therapy parameters, perform a therapy session, collect user satisfaction data for the therapy session, if the user satisfaction data meets a threshold, complete the trial period with the first set of therapy parameters, provide the first set of therapy parameters, kinematic data sensed by the one or more sensors, and the satisfaction data to a learning algorithm, receive optimized therapy parameters from the learning algorithm, the learning algorithm predicting a modified outcome based on an individual database which includes an aggregation of kinematic data and satisfaction data from multiple therapy sessions for the user, and perform another therapy session with the optimized therapy parameters. Wherein, optionally, the device is a wearable transcutaneous device.

In some embodiments, the sets of therapy parameters are based on kinematic data and satisfaction data for a plurality of users.

In some embodiments, the one or more hardware processors are further configured to select a second set from the sets of therapy parameters and perform another therapy session with the second set during the trial period.

In some embodiments, the threshold is predetermined and/or selected by the user. In some embodiments, the trial period comprises multiple days (e.g., 5, 10, 15 or more days).

In several embodiments, the neurostimulation devices, systems and methods disclosed herein are connected, coupled and/or in communication with a second device (and two or more additional devices). The second (or additional devices) can include, for example, a tablet, phone, smartwatch, computing device, display etc.

In some embodiments, optimized therapy parameters are based at least in part on responses from a plurality of user and/or a plurality of sets of therapy parameters.

In some embodiments, responses include satisfaction data and kinematic data.

In some embodiments, disclosed herein is a neurostimulation device for stimulating one or more peripheral nerves of a user. In some embodiments, the device includes (for example, comprises, consists, or consists essentially of) one or more electrodes configured to generate electric stimulation signals based on therapy parameters, one or more sensors configured to detect motion signals, wherein the one or more sensors are operably connected to the neurostimulation device, and one or more hardware processors configured to receive a set of therapy parameters, perform a therapy session with the set of therapy parameters, collect kinematic and user satisfaction data for the therapy session, provide the set of therapy parameters, the kinematic data, and the user satisfaction data to a learning algorithm, receive optimized therapy parameters from the learning algorithm, the learning algorithm predicting a modified outcome based on an individual database which includes an aggregation of kinematic data and satisfaction data from multiple therapy sessions for the user, and perform another therapy session with the optimized therapy parameters. Optionally, the device is a wearable transcutaneous device.

In some embodiments, disclosed herein is a base station for a neurostimulation device. The neurostimulation device stimulates one or more peripheral nerves of a user. The base station includes (for example, comprises, consists, or consists essentially of) one or more hardware processors configured to receive kinematic data, satisfaction data, and/or therapy parameters for a therapy session, provide the kinematic data, satisfaction data, and/or therapy parameters to a learning algorithm, receive optimized therapy parameters from the learning algorithm, the learning algorithm predicting a modified outcome based on an individual database which includes an aggregation of kinematic data and satisfaction data from multiple therapy sessions for the user, and provide the optimized therapy parameters to the wearable neurostimulation device. Optionally the device is a wearable transcutaneous device.

In some embodiments, disclosed herein is a neuromodulation device for modulating one or more nerves of a user. In some embodiments, the device includes (for example, comprises, consists, or consists essentially of) means configured to generate neuromodulation, sensing means, wherein the sensing means are operably connected to the neuromodulation device. The device can further include one or more hardware processors configured to perform a therapy session with therapy parameters, measure data from the sensing means, adjust the therapy parameters based on the measured data, optimize the adjusted therapy parameters (i) based on an aggregation or collection of the measured data taken over at least three different therapy sessions of the same patient and/or (ii) based on an aggregation or collection of data taken from multiple different patients.

In some embodiments, the optimized therapy parameters are based at least in part on responses from a plurality of user. The responses can include satisfaction data and kinematic data. The optimized therapy parameters can include a plurality of sets of therapy parameters. The set of therapy parameters are based, in one embodiment, at least in part on a predetermined decision tree and/or one or more user profiles.

In some embodiments, the kinematic data is collected from a sensor or multiple sensors onboard the neurostimulation device or elsewhere. The kinematic data can include raw accelerometer and/or gyroscope data.

In some embodiments, the one or more sensors are further configured to employ an aggregation learning algorithm to receive the optimized therapy parameters.

In some embodiments, the device includes three to six or more electrodes (e.g., 3, 4, 5, 6), and is partially implantable or is entirely transcutaneous. Several embodiments provide a wrist worn or ear worn device, or both.

In some embodiments, the aggregation or collection of data taken from multiple different patients may be taken from subpopulations of patients with like characteristics to the treated patient, and wherein the subpopulations may be groups of patients with similar age ranges, gender, hormone levels, drugs used, and/or tremor characteristics.

In some embodiments, the use of the device is for the treatment of depression (including but not limited to post-partum depression, depression affiliated with neurological diseases, major depression, seasonal affective disorder, depressive disorders, etc.), inflammation, Lyme disease, stroke, neurological diseases (such as Parkinson's and Alzheimer's), and gastrointestinal issues (including those in Parkinson's disease).

In some embodiments, the device is used for the treatment of inflammatory bowel disease (such as Crohn's disease), rheumatoid arthritis, multiple sclerosis, psoriatic arthritis, osteoarthritis, psoriasis and other inflammatory diseases.

In some embodiments, the device is used for the treatment of inflammatory skin conditions.

In some embodiments, the device is used for the treatment of chronic fatigue syndrome.

In some embodiments, the device is used for the treatment of chronic inflammatory symptoms and flare ups.

In some embodiments, the device is used for the treatment of cardiac conditions (such as atrial fibrillation).

In some embodiments, the device is used for the treatment of immune dysfunction.

In some embodiments, the device is used to stimulate the autonomic nervous system.

In some embodiments, the device is used to balance the sympathetic/parasympathetic nervous systems.

In some embodiments, the device is used in a system and/or method which further comprises a wrist worn, leg worn or head (e.g., ear) worn device.

In some embodiments, the device is used to reduce the dose of one or more drugs or pharmacological agents delivered to a patient, wherein the dose may for example include the amount, duration, number or frequency of the drug or pharmacological agent.

In some embodiments, the device is used to enhance the efficacy of one or more drugs or pharmacological agents delivered to a patient.

In some embodiments, disclosed herein is a wearable neurostimulation device for transcutaneously stimulating one or more peripheral nerves of a user to improve diagnosis, prognosis, and/or therapeutic outcomes for a disease. In some embodiments, the device includes (for example, comprises, consists, or consists essentially of) one or more electrodes configured to generate electric stimulation signals based on therapy parameters, the therapy parameters being selected from a plurality of predefined profiles determined by an aggregation learning algorithm, the aggregation learning algorithm predicting a plurality of outcomes for the user based on a plurality of predefined profiles, the predefined profiles being based on features extracted from data for a plurality of users, one or more sensors configured to detect physiological data, wherein the one or more sensors are operably connected to the wearable neurostimulation device. The device further includes one or more hardware processors configured to perform a therapy session with the therapy parameters, measure physiological data from the one or more sensors during the therapy session, wherein the physiological data optionally includes one or more of heart rate, blood glucose, blood pressure, respiration rate, body temperature, blood volume, sound pressure, photoplethysmography, electroencephalogram, electrocardiogram, blood oxygen saturation, and/or skin conductance data, adjust the therapy parameters based on the measured physiological data from the therapy session, optimize the adjusted therapy parameters by a learning algorithm based on an individual database, the learning algorithm predicting a modified outcome based on the individual database which includes an aggregation of physiological data and satisfaction data from multiple therapy sessions for the user.

In some embodiments, the physiological data includes respiration rate and heart rate, and wherein the disease is depression.

In some embodiments, the one or more sensors are further configured to detect sleep patterns and activity level of the user.

In some embodiments, the disease is migraine or Lyme.

In some embodiments, disclosed herein is a neurostimulation device for stimulating one or more peripheral nerves of a user to improve diagnosis, prognosis, and/or therapeutic outcomes for a disease. In some embodiments, the device includes (for example, comprises, consists, or consists essentially of) one or more electrodes configured to generate electric stimulation signals based on therapy parameters and one or more sensors configured to detect physiological data, wherein the one or more sensors are operably connected to the neurostimulation device. The device can further include one or more hardware processors configured to receive a set of therapy parameters, perform a therapy session with the set of therapy parameters, collect physiological and user satisfaction data for the therapy session, wherein the physiological data optionally includes one or more of heart rate, blood glucose, blood pressure, respiration rate, body temperature, blood volume, sound pressure, photoplethysmography, electroencephalogram, electrocardiogram, blood oxygen saturation, and/or skin conductance data, provide the set of therapy parameters, the physiological data, and the user satisfaction data to a learning algorithm, receive optimized therapy parameters from the learning algorithm, the learning algorithm predicting a modified outcome based on an individual database which includes an aggregation of physiological data and satisfaction data from multiple therapy sessions for the user, and perform another therapy session with the optimized therapy parameters. Wherein, optionally, the device is a wearable transcutaneous device.

In some embodiments, the physiological data includes respiration rate and heart rate, and wherein the disease is depression.

In some embodiments, the one or more sensors are further configured to detect sleep patterns and activity level of the user.

In some embodiments, the disease is migraine or Lyme.

In some embodiments, disclosed herein is a neuromodulation device for modulating one or more nerves of a user to improve diagnosis, prognosis, and/or therapeutic outcomes for a disease. In some embodiments, the device includes (for example, comprises, consists, or consists essentially of) means configured to generate neuromodulation and sensing means for detecting physiological data, wherein the sensing means are operably connected to the neuromodulation device. The device can further include one or more hardware processors configured to perform a therapy session with therapy parameters, measure physiological data from the sensing means for the therapy session, wherein the physiological data optionally includes one or more of heart rate, blood glucose, blood pressure, respiration rate, body temperature, blood volume, sound pressure, photoplethysmography, electroencephalogram, electrocardiogram, blood oxygen saturation, and/or skin conductance data, adjust the therapy parameters based on the measured data, optimize the adjusted therapy parameters (i) based on an aggregation or collection of the physiological data taken over at least three different therapy sessions of the same patient and/or (ii) based on an aggregation or collection of physiological data taken from multiple different patients.

In some embodiments, the physiological data includes respiration rate and heart rate, and wherein the disease is depression.

In some embodiments, the sensing means is further configured to detect sleep patterns and activity level of the user.

In some embodiments, the disease is migraine or Lyme.

In some embodiments, disclosed herein is a method of neuromodulating one or more nerves. The method can include (for example, comprise, consist, or consist essentially of) selecting from a plurality of predefined profiles of therapy parameters that are associated with a plurality of outcomes for the user, the predefined profiles being based on features extracted from data for a plurality of users, detecting motion signals during a therapy session employing the therapy parameters, measuring kinematic data including data from the detected motion signals during the therapy session, wherein the data optionally includes tremor data, adjusting the therapy parameters based on measured data from the detected motion signals during the therapy session, optimizing the adjusted therapy parameters based on an individual database, and predicting a modified outcome based on the individual database which includes an aggregation of kinematic data and satisfaction data from multiple therapy sessions for the user.

In some embodiments, disclosed herein is a method of neuromodulating one or more nerves. In some embodiments, the method includes (for example, comprises, consists, or consists essentially of) performing a therapy session with therapy parameters, collecting passive kinematic data throughout the day of the therapy session, the passive kinematic data being indicative of activities performed by the user at times outside of the therapy session, comparing the passive kinematic data to a predicted user satisfaction database, the predicted user satisfaction database including an aggregation of passive kinematic data from multiple therapy sessions, and determining a user satisfaction level for the therapy session based on the comparison. In some embodiments, the method further includes collecting test kinematic data during the therapy session, comparing the test kinematic data to a predicted user test satisfaction database, the predicted user test satisfaction database including an aggregation of test kinematic data from multiple therapy sessions for the user, and determining a user test satisfaction level for the therapy session based on the comparison.

In some embodiments, the features extracted from the data include test kinematic data and/or satisfaction data.

In some embodiments, the therapy parameters are based at least in part on a predetermined decision tree and/or one or more user profiles.

In some embodiments, the kinematic data is collected from a sensor onboard the neurostimulation device or elsewhere. The kinematic data can include raw accelerometer and/or gyroscope data.

In some embodiments, the features extracted from the data for the plurality of users include one or more of geospatial data, temporal data, disease, patient attributes or characteristics, or neurostimulation device characteristics associated with the extracted features.

In some embodiments, the individual database includes kinematic data and/or satisfaction data accumulated over multiple hours/days/weeks/months.

In some embodiments, the method comprises sensing via sensors that employ an aggregation learning algorithm to determine the user satisfaction level and/or a learning algorithm to determine the user satisfaction level.

In some embodiments, the devices or methods are used for treatment of disorders and/or associated symptoms such as depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). In some embodiments, conditions such as stroke, cardiac events, inflammation, etc. are treated.

In several embodiments, one or more of bradykinesia, dyskinesia, gait dysfunction, dystonia and/or rigidity are treated with the devices and methods described herein (e.g., in connection with Parkinson's disease or in connection with other disorders). Rehabilitation of movement is treated in some embodiments (for example to restore or improve movement and motion) in subjects who have suffered from an acute or chronic event, including, for example, cardiac events (such as atrial fibrillation, hypertension, epilepsy, and stroke), inflammation, neuroinflammation, etc. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One, two, three or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection are treated, including for example, inflammation (e.g., neuroinflammation), headache, back pain, joint pain and stiffness, muscle pain and tension, etc.

### BRIEF DESCRIPTION OF THE DRAWINGS

The following drawings are for illustrative purposes only and show non-limiting embodiments. Features from different figures may be combined in several embodiments.

FIG. 1A illustrates a block diagram of an example neuromodulation (e.g., neurostimulation) device.

FIG. 1B illustrates a block diagram of an embodiment of a controller that can be implemented with the hardware components described with respect to FIG. 1A.

FIG. 1C schematically illustrates an embodiment of a neuromodulation device and base station.

FIG. 2 illustrates a block diagram of an embodiment of a controller that can be implemented with the hardware components described with respect to FIGS. 1A-C.

FIG. 3 illustrates a flow chart of an embodiment of a process for personalization of a neurostimulation device.

FIG. 4 schematically illustrates an embodiment of a system for personalization of the neurostimulation device employing a learning algorithm in the base station.

FIG. 5 schematically illustrates an embodiment of a system for personalization of the neurostimulation device employing a learning algorithm in the cloud.

FIG. 6 schematically illustrates an embodiment of a system for personalization of the neurostimulation device employing a learning algorithm in the neuromodulation device.

FIG. 7 schematically illustrates an embodiment of a system for personalization of the neurostimulation device employing a learning algorithm in a third party device.

FIG. 8 illustrates a flow chart of a non-limiting embodiment of a process for aggregating satisfaction data.

FIG. 9 illustrates a flow chart of a non-limiting embodiment of a process for personalizing therapeutic protocols employing the aggregated satisfaction data from FIG. 8.

FIG. 10 illustrates a flow chart of a non-limiting embodiment of a process for aggregating data collected from an individual including kinematic and satisfaction data.

FIG. 11 illustrates a flow chart of a non-limiting embodiment of a process for personalizing therapeutic protocols employing the aggregated satisfaction data from FIG. 10.

FIG. 12 illustrates a flow chart of a non-limiting embodiment of a process for personalizing therapeutic protocols employing a decision tree during a trial period and a learning algorithm.

FIG. 13 illustrates a flow chart of a non-limiting embodiment of a process performed by the neurostimulation device employing a learning algorithm.

FIG. 14 illustrates a flow chart of a non-limiting embodiment of a process performed by the base station or third part device employing the learning algorithm.

FIG. 15 illustrates a flow chart of a non-limiting embodiment of a process for aggregating data.

FIG. 16 illustrates a flow chart of a non-limiting embodiment of a process for personalizing therapeutic protocols employing the aggregated data from FIG. 15.

FIG. 17 is a table of sample parameters of response profiles that can be employed by the system disclosed herein.

FIG. 18 illustrates a flow chart of a non-limiting embodiment of a machine learning process employed by the methods disclosed in any of FIGS. 12-15.

FIG. 19 illustrates an overall patient study to develop treatment algorithms.

FIG. 20 is a graph of patient tremor improvement ratios for different waveforms based on measured kinematic data of the patient.

FIG. 21 is a bar chart of patient activities of daily living (ADL) scores for different waveforms based on subjective data from the patients in the study.

FIG. 22 is a bar chart of patient tremor improvement ratios for different waveforms based on measured kinematic data for individual patients in the study.

FIG. 23 is a bar chart of potential patient tremor improvement ratios for TAPS vs. the best waveform from the study.

FIG. 24 includes bar charts of potential ADL scores for TAPS and the best waveform for both cohorts 1 and 2.

FIG. 25 illustrates an embodiment of a method for selecting a stimulation output.

FIG. 26 illustrates a proposed timing for a method that a training or preview window and an algorithm evaluation period.

FIG. 27 illustrates the ADL scores for TAPS and the best waveform for both cohorts 1 and 2 that are associated with the best ADL scores.

FIG. 28 illustrates the ADL scores for TAPS and the best waveform for cohort 1 that is associated with the best kinematic data.

FIG. 29 illustrates healthcare domains where machine learning, including reinforcement learning, disclosed herein can be implemented.

FIG. 30 schematically illustrates a reinforcement learning system represented by an agent which can be employed in the systems disclosed herein.

FIG. 31 illustrates a reinforcement learning algorithm performed by the agent of FIG. 30.

FIG. 32 illustrates an engineering implementation of the machine learning algorithm of FIGS. 30 and 31.

### DETAILED DESCRIPTION

Disclosed herein are devices configured for providing neuromodulation (e.g., neurostimulation). The neuromodulation (e.g., neurostimulation) devices provided herein may be configured to stimulate peripheral nerves of a user. The neuromodulation (e.g., neurostimulation) devices may be configured to transmit one or more neuromodulation (e.g., neurostimulation) signals across the skin of the user. In many embodiments, the devices are wearable devices configured to be worn by a user. The user may be a human, another mammal, or other animal user. The system could also include signal processing systems and methods for enhancing diagnostic and therapeutic protocols relating to the same. In some embodiments, the device is configured to be wearable on an upper extremity of a user (e.g., a wrist, forearm, arm, and/or finger(s) of a user). In some embodiments, the device is configured to be wearable on a lower extremity (e.g., ankle, calf, knee, thigh, foot, and/or toes) of a user. In some embodiments, the device is configured to be wearable on the head or neck (e.g., forehead, ear, neck, nose, and/or tongue). In several embodiments, dampening or blocking of nerve impulses and/or neurotransmitters are provided. In some embodiments, nerve impulses and/or neurotransmitters are enhanced. In some embodiments, the device is configured to be wearable on or proximate an ear of a user, including but not limited to auricular neuromodulation (e.g., neurostimulation) of the auricular branch of the vagus nerve, for example. One or more of the vagus, tragus, trigeminal or cranial nerves may be treated in some embodiments. The device could be unilateral or bilateral, including a single device or multiple devices connected with wires or wirelessly. Transcutaneous neuromodulation is provided in several embodiments, although subcutaneous and percutaneous components may also be used. In some embodiments, the device includes three to six or more electrodes (e.g., 3, 4, 5, 6), and is partially implantable or is entirely transcutaneous.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, greater auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

Single or multiple bands that partially or fully encircle a limb (such as a wrist, ankle, arm, leg) are provided in some embodiments. Ear devices are also provided in some embodiments that can be used with or without a limb band. In one embodiment, an ear device and a wrist band are provided for synergistic treatment. An auricular (e.g., ear) device can include an earpiece or bud for one or more portions of the ear such as an ear canal or external ear. One to six or more electrodes may be placed on the earpiece or bud, or on a device connected to the earpiece/bud. Right, left or two earpieces are provided in some embodiments.

Systems with compact, ergonomic form factors are needed to enhance efficacy, compliance, and/or comfort when using non-invasive or wearable neuromodulation devices. In several embodiments, neuromodulation systems and methods are provided that enhance or inhibit nerve impulses and/or neurotransmission, and/or modulate excitability of nerves, neurons, neural circuitry, and/or other neuroanatomy that affects activation of nerves and/or neurons. For example, neuromodulation (e.g., neurostimulation) can include one or more of the following effects on neural tissue: depolarizing the neurons such that the neurons fire action potentials; hyperpolarizing the neurons to inhibit action potentials; depleting neuron ion stores to inhibit firing action potentials; altering with proprioceptive input; influencing muscle contractions; affecting changes in neurotransmitter release or uptake; and/or inhibiting firing.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz, 100 Hz, 150 Hz, 200 Hz, in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

Stimulation of peripheral nerves can provide therapeutic benefit across a variety of diseases, including but not limited to disorders and/or associated symptoms such as movement disorders (including but not limited to essential tremor, Parkinson's tremor, orthostatic tremor, and multiple sclerosis), urological disorders, gastrointestinal disorders, cardiac diseases, inflammatory diseases, mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, epilepsy, and stroke) are treated in one embodiment. Inflammatory skin conditions and immune dysfunction are also treated in some embodiments. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s).

In several embodiments, one or more of bradykinesia, dyskinesia, gait dysfunction, dystonia and/or rigidity are treated. These may be treated in connection with Parkinson's disease or in connection with other disorders. Rehabilitation of movement is treated in some embodiments (for example to restore or improve movement and motion) in subjects who have suffered from an acute or chronic event including, for example, cardiac events (such as atrial fibrillation, hypertension, epilepsy, and stroke), inflammation, neuroinflammation, etc. Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation), headache, back pain, joint pain and stiffness, muscle pain and tension, etc.

In some embodiments, wearable systems and methods as disclosed herein can advantageously be used to identify whether a treatment is effective in significantly reducing or preventing a medical condition, including but not limited to tremor severity. Although tremor is treated in several embodiments, the devices described herein are used to treat conditions other than tremor. Wearable sensors can advantageously monitor, characterize, and aid in the clinical management of hand tremor as well as other medical conditions including those disclosed elsewhere herein. Not to be limited by theory, clinical ratings of medical conditions, e.g., tremor severity can correlate with simultaneous measurements of wrist motion using inertial measurement units (IMUs). For example, tremor features extracted from IMUs at the wrist can provide characteristic information about tremor phenotypes that may be leveraged to improve diagnosis, prognosis, and/or therapeutic outcomes. Kinematic measures can correlate with tremor severity, and machine learning algorithms incorporated in neuromodulation systems and methods as disclosed for example herein can predict tremor severity.

In other non-tremor embodiments, physiological data including heart rate, blood glucose, blood pressure, respiration rate, body temperature, blood volume, sound pressure, photoplethysmography, electroencephalogram, electrocardiogram, blood oxygen saturation, and/or skin conductance as well as patient data from third party devices can be collected and/or aggregated to improve diagnosis, prognosis, and/or therapeutic outcomes for migraine, depression, and/or Lyme disease. For example, physiological data including respiration rate and heart rate along with data related to sleep patterns and activity level can be collected and/or aggregated to improve diagnosis, prognosis, and/or therapeutic outcomes for depression.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc.

### Neuromodulation Devices

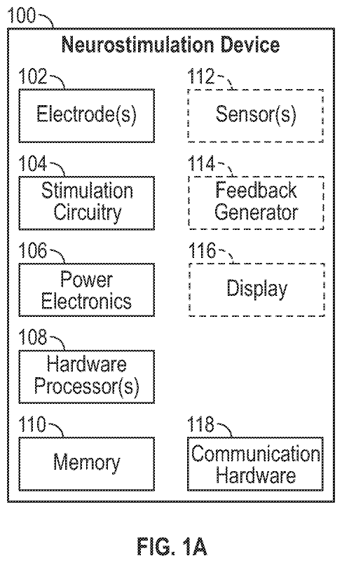

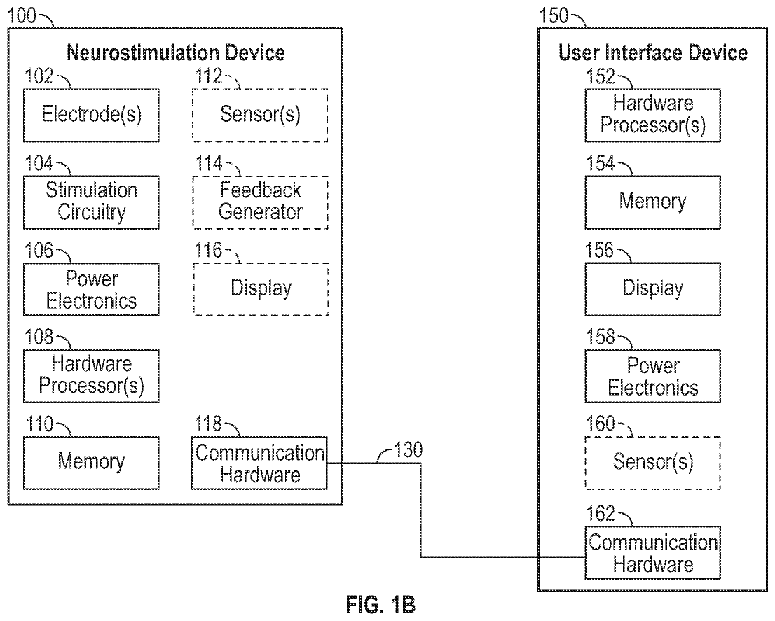

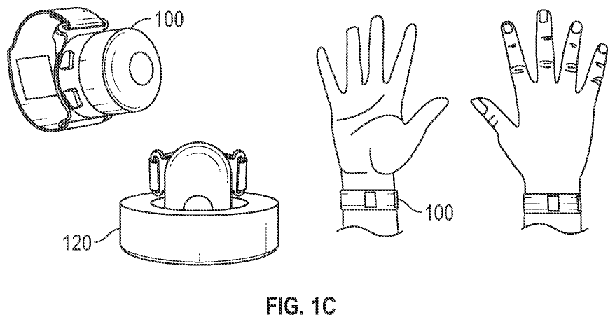

FIG. 1A illustrates a block diagram of an example neuromodulation (e.g., neurostimulation) device 100. The neurostimulation device 100 includes multiple hardware components which are capable of or programmed to provide therapy across the skin of the user. As illustrated in FIG. 1A, some of these hardware components may be optional as indicated by dashed blocks. In some instances, the neurostimulation device 100 may only include the hardware components that are required for stimulation therapy. The hardware components are described in more detail below.

The neurostimulation device 100 can include two or more effectors, e.g. electrodes 102 for providing neurostimulation signals. For example, 2-12 effectors (such as electrodes) are provided (e.g., 2, 3, 4, 5, 6, 7, 8, 9, 10, 11, 12, or more). In some instances, the neurostimulation device 100 is configured for transcutaneous use only and does not include any percutaneous or implantable components. In some embodiments, the electrodes can be dry electrodes. In some embodiments, water or gel can be applied to the dry electrode or skin to improve conductance. In some embodiments, the electrodes do not include any hydrogel material, adhesive, or the like.

In some embodiments 3-12 or more electrodes are used (e.g. 3, 6, 9 or 12). In one embodiment, none of the electrodes are in contact with areas that cause discomfort. The electrodes could be percutaneous or microneedle electrodes in other embodiments, or only transcutaneous (e.g., not percutaneous, microneedles, or implanted electrodes in some embodiments). In many embodiments, the transcutaneous device is a wearable band or earpiece. The band may partially or fully surround a wrist, finger, arm, leg, ankle or head. Patches may be used, but in many embodiments a patch is not used.

The neurostimulation device 100 can further include stimulation circuitry 104 for generating signals that are applied through the electrode(s) 102. In some embodiments, the neurostimulation device 100 employs three or more electrodes 102 to apply a stimulation signal to the patient. For example, in some embodiments, at least one electrode is redundant to another electrode (e.g., 2 or more redundant common electrodes and/or 2 or more redundant stimulation electrodes). In this way, even if the electrical contact between one of the two electrodes and the patient's skin is poor increasing resistance, the electrical contact between the redundant electrode and the patient's skin can complete the electrical circuit with a normal or expected level of resistance.

In some embodiments, the 2 or more common electrodes and/or 2 or more stimulation electrodes are circumferentially spaced about the band so that even if the band rotates slightly on the wrist causing an electrode to lose contact with the patient's skin, the redundant electrode will still be in contact with the patient's skin to compete the circuit with a normal or expected level of resistance. In this way, the desired stimulation signal (e.g., frequency, phase, timing, amplitude, and/or offsets) is applied to the patient even when the band rotates on the patient's wrist. The band is less sensitive to electrical contact variations between the electrodes and the patient's skin caused by variations in the angular orientation of the band on the wrist.

The signals can vary in frequency, phase, timing, amplitude, or offsets. The neurostimulation device 100 can also include power electronics 106 for providing power to the hardware components. For example, the power electronics 106 can include a battery.

The neurostimulation device 100 can include one or more hardware processors 108. The hardware processors 108 can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. In an embodiment, all of the processing discussed herein is performed by the hardware processor(s) 108. The memory 110 can store data specific to patient and rules as discussed below.

In the illustrated figure, the neurostimulation device 100 can include one, two, three, or more sensors 112 which can include any number of combination of inertial measurement units (IMUs) single or multi-axis accelerometers, gyroscopes, inclinometers (to measure and correct for changes in the gravity field resulting from slow changes in the device's orientation), magnetometers; fiber optic electro goniometers, optical tracking or electromagnetic tracking; electromyography (EMG) to detect firing of tremoring muscle; electroneurogram (ENG) signals; cortical recordings by techniques such as electroencephalography (EEG) or direct nerve recordings on an implant in close proximity to the nerve; heart rate or HRV sensors, galvanic skin response sensors (GSR), thermocouples, photoplethysmography sensor (PPG), temperature sensors (e.g., for body/skin temperature or ambient temperature), and/or other physiologic sensors, for example. In some embodiments, the one or more sensors can be employed to measure response to therapy as well as to calibrate therapy. As shown in the figure, the sensor(s) 112 may be optional.

In some embodiments, the one or more sensors 112 include an IMU. In some embodiments, the IMU can include one or more of a gyroscope, accelerometer, and magnetometer. The IMU can be affixed or integrated with the neuromodulation (e.g., neurostimulation) device 100. In an embodiment, the IMU is an off the shelf component. In addition to its ordinary meaning, the IMU can also include specific components as discussed below. For example, the IMU can include one more sensors capable of collecting motion data. In an embodiment, the IMU includes an accelerometer. In some embodiments, the IMU can include multiple accelerometers to determine motion in multiple axes. Furthermore, the IMU can also include one or more gyroscopes and/or magnetometer in additional embodiments. Since the IMU can be integrated with the neurostimulation device 100, the IMU can generate data from its sensors responsive to motion, movement, or vibration felt by the neurostimulation device 100. Furthermore, when the neurostimulation device 100 with the integrated IMU is worn by a user, the IMU can enable detection of voluntary and/or involuntary motion of the user.

The neurostimulation device 100 can optionally include user interface components, such as a feedback generator 114 and a display 116. The display 116 can provide instructions or information to users relating to calibration or therapy. The display 116 can also provide alerts, such an indication of response to therapy, for example. Alerts may also be provided using the feedback generator 114, which can provide haptic feedback to the user, such as upon initiation or termination of stimulation, for reminder alerts, to alert the user of a troubleshooting condition, to perform a tremor inducing activity to measure tremor motion, among others. Accordingly, the user interface components, such as the feedback generator 114 and the display 116 can provide audio, visual, and haptic feedback to the user.

Furthermore, the neurostimulation device 100 can include communications hardware 118 for wireless or wired communication between the neurostimulation device 100 and an external system, such as the user interface device 150 discussed below. The communications hardware 118 can include an antenna. The communications hardware 118 can also include an Ethernet or data bus interface for wired communications.

While the illustrated figure shows several components of the neurostimulation device 100, some of these components are optional and not required in all embodiments of the neurostimulation device 100. In some embodiments, a system can include a diagnostic device or component that does not include neuromodulation functionality. The diagnostic device could be a companion wearable device connected wirelessly through a connected cloud server, and include, for example, sensors such as cardiac activity, skin conductance, and/or motion sensors as described elsewhere herein.

In some embodiments, the neurostimulation device 100 can also be configured to deliver one, two or more of the following: magnetic, vibrational, mechanical, thermal, ultrasonic, or other forms of stimulation instead of, or in addition to electrical stimulation. Such stimulation can be delivered via one, two, or more effectors in contact with, or proximate the skin surface of the patient. However, in some embodiments, the device is configured to only deliver electrical stimulation, and is not configured to deliver one or more of magnetic, vibrational, mechanical, thermal, ultrasonic, or other forms of stimulation.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

### User Interface Devices

FIG. 1B illustrates communications between the neurostimulation device 100 and a user interface device 150 over a communication link 130. The communication link 130 can be wired or wireless. The neuromodulation (e.g., neurostimulation) device 100 is capable of communicating and receiving instructions from the user interface device 150. The user interface device 150 can include a computing device. In some embodiments, the user interface device 150 is a mobile computing device, such as a mobile phone, a smartwatch, a tablet, or a wearable computer. The user interface device 150 can also include server computing systems that are remote from the neurostimulation device. The user interface device 150 can include hardware processor(s) 152, a memory 154, display 156, and power electronics 158. In some embodiments, a user interface device 150 can also include one or more sensors 160, such as sensors described elsewhere herein. Furthermore, in some instances, the user interface device 150 can generate an alert responsive to device issues or a response to therapy. The alert may be received from the neurostimulation device 100.

In additional embodiments, data acquired from the one or more sensors 112 is processed by a combination of the hardware processor(s) 108 and hardware processor(s) 152. In further embodiments, data collected from one or more sensors 112 is transmitted to the user interface device 150 with little or no processing performed by the hardware processors 108. In some embodiments, the user interface device 150 can include a remote server that processes data and transmits signals back to the neurostimulation device 100 (e.g., via the cloud 122).

FIG. 1C schematically illustrates a neurostimulation device 100 and base station 120. The neurostimulation device 100 can include a stimulator and detachable band including two or more working electrodes (positioned over the median and radial nerves) and a counter-electrode positioned on the dorsal side of the wrist. The electrodes could be, for example, dry electrodes or hydrogel electrodes. The base station 120 can be configured to stream movement sensor and usage data on a periodic basis, e.g., daily and charge the neurostimulation device 100. The neurostimulation device 100 stimulation bursting frequency can be calibrated to a lateral postural hold task “wing-beating” or forward postural hold task for a predetermined time, e.g., 10, 20, 30 seconds for each subject. Other non-limiting examples of neurostimulation device 100 parameters can be as disclosed elsewhere herein.

In some embodiments, stimulation may alternate between each nerve such that the nerves are not stimulated simultaneously. In some embodiments, all nerves are stimulated simultaneously. In some embodiments, stimulation is delivered to the various nerves in one of many bursting patterns. The stimulation parameters may include on/off, time duration, intensity, pulse rate, pulse width, waveform shape, and the ramp of pulse on and off. In one embodiment the pulse rate may be from about 1 to about 5000 Hz, about 1 Hz to about 500 Hz, about 5 Hz to about 50 Hz, about 50 Hz to about 300 Hz, or about 150 Hz, and overlapping ranges therein. In some embodiments, the pulse rate may be from 1 kHz to 20 KHz. In some embodiments, a pulse width may range from, in some cases, 50 to 500 μs (micro-seconds), such as approximately 50-150,150-300, 300-500, such as 100, 200, 300, 400 μs, and overlapping ranges therein. Although frequencies below 5 kHz are used in several embodiments, some embodiments use higher frequency stimulation (e.g., of nerves at or near the wrist or ear) of 5-75 kHz (e.g., 10-40 KHz, 15-60 kHz, etc.) and a pulse width of 1-20, 10-50, 10-40 μs. The intensity of the electrical stimulation may vary from 0 mA to 500 mA, and a current may be approximately 1-11, 1-20, 5-50, 10-100 mA, and overlapping ranges therein. The electrical stimulation can be adjusted in different patients and with different methods of electrical stimulation. The increment of intensity adjustment may be, for example, 0.1 mA to 1.0 mA, such as 0.1-0.5, 0.5-0.75, 5-1 mA, and overlapping ranges therein. In some embodiments, the stimulation may last for approximately 10 minutes to 1 hour, such as approximately 10, 20, 30, 40, 50, or 60 minutes, or ranges including any two of the foregoing values. In some embodiments, stimulation may be provided for 30, 40, 50, 60, 80, 90, 120, 150 minutes 1-4 times a day. In some embodiments, stimulation occurs for 2-15 minutes (e.g., 3, 5, 7, 10 minutes) every hour (or on another interval) for a total of 40-240 minutes (e.g., 60, 80, 90, 120, 150 minutes) in a 12- or 24-hour period. Differing dosing schedules and/or differing stimulation parameters may reduce tolerance or habituation and/or may increase patient comfort/compliance. In one embodiment, beneficial effects of stimulation are provided during off periods; for example, a patient's tremor or other symptom/indication is reduced because the prior stimulation results in a prolonged effect on the nerve(s). Thus, a patient may be able to reduce the length, duration etc. of therapy over time. In some embodiments, a plurality of electrical stimuli can be delivered offset in time from each other by a predetermined fraction of multiple of a period of a measured rhythmic biological signal such as hand tremor, such as about ¼, ½, or ¾ of the period of the measured signal for example. Further possible stimulation parameters are described, for example, in U.S. Pat. No. 9,452,287 to Rosenbluth et al., U.S. Pat. No. 9,802,041 to Wong et al., PCT Pub. No. WO 2016/201366 to Wong et al., PCT Pub. No. WO 2017/132067 to Wong et al., PCT Pub. No. WO 2017/023864 to Hamner et al., PCT Pub. No. WO 2017/053847 to Hamner et al., PCT Pub. No. WO 2018/009680 to Wong et al., and PCT Pub. No. WO 2018/039458 to Rosenbluth et al., each of the foregoing of which are hereby incorporated by reference in their entireties.

### Controller

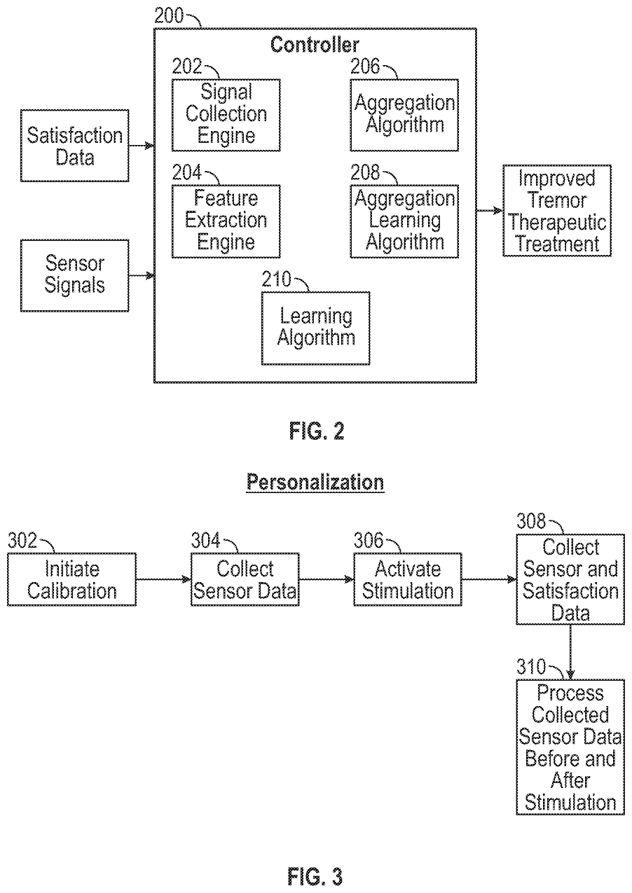

FIG. 2 illustrates a block diagram of an embodiment of a controller 200 that can be implemented with the hardware components described above with respect to FIGS. 1A-1C. The controller 200 can include multiple engines for performing the processes and functions described herein. The engines can include programmed instructions for performing processes as discussed herein for detection of input conditions, processing data, and control of output conditions. The engines can be executed by the one or more hardware processors of the neuromodulation (e.g., neurostimulation) device 100 alone or in combination with the base station 150, the user interface device 150, and/or the cloud 122. The programming instructions can be stored in a memory 110. The programming instructions can be implemented in C, C++, JAVA, or any other suitable programming languages. In some embodiments, some or all of the portions of the controller 200 including the engines can be implemented in application specific circuitry such as ASICs and FPGAs. Some aspects of the functionality of the controller 200 can be executed remotely on a server (not shown) over a network. While shown as separate engines, the functionality of the engines as discussed below is not necessarily required to be separated. Accordingly, the controller 200 can be implemented with the hardware components described above with respect to FIGS. 1A-1C.

The controller 200 can include a signal collection engine 202. The signal collection engine 202 can enable acquisition of raw data from the sensors 112 embedded in the device 100 as well as patient satisfaction data. The sensor data can include but is not limited to accelerometer or gyroscope data from the IMU. In some embodiments, the sensor data can include test kinematic data taken during a therapy session. In some embodiments, the sensor data can include passive kinematic data. Passive kinematic data is data collected at times outside of the therapy session.

In some embodiments, the neuromodulation, e.g., neurostimulation device 100 or the user interface device 150 with sensors can collect kinematic or motion data (test and/or passive data), or data from other sensors, can measure data over a longer period of time, for example 1, 2, 3, 4, 5, 10, 20, 30 weeks, 1, 2, 3, 6, 9, 12 months, or 1, 2, 3, 5, 10 years or more or less, or ranges incorporating any two of the foregoing values, to determine features, or biomarkers, associated with the onset of tremor diseases, such as essential tremor, Parkinson's disease, dystonia, multiple sclerosis, etc. Biomarkers could include specific changes in one or more features of the data over time, or one or more features crossing a predetermined threshold. In some embodiments, features of tremor inducing tasks have been stored on the neurostimulation device 100 and used to automatically activate sensors when those tremor inducing tasks are being performed, to measure and store data to memory during relevant times.

The satisfaction data can include but is not limited to subjective data provided by the patient. The subjective data can relate to pre or post treatment and/or patient activities of daily living (ADL). In some embodiments, the patient inputs a value that reflects a level of satisfaction. The level of satisfaction can be selected from a predetermined range. In some embodiments, the range is from 1 to 4. Of course, the range can be any range and is not limited to 1 to 4.

In some embodiments, the signal collection engine 202 can also perform signal preprocessing on the raw data. Signal preprocessing can include noise filtering, smoothing, averaging, and other signal preprocessing techniques to clean the raw data. In some embodiments, portions of the signals can be discarded by the signal collection engine 202. In some embodiments, portions of the signals are associated with a time stamp or other temporal indicator.

The controller 200 can also include a feature extraction engine 204. The feature extraction engine 204 can extract relevant features from the signals, including from the satisfaction data, collected by the signal collection engine 202. The features can be in time domain and/or frequency domain. For example, some of the features can include amplitude, bandwidth, area under the curve (e.g., power), energy in frequency bins, peak frequency, ratio between frequency bands, and the like. The features can be extracted using signal processing techniques such as Fourier transform, band pass filtering, low pass filtering, high pass filtering and the like.

The controller 200 can further include an aggregation algorithm 206. The aggregation algorithm 206 can collect the extracted features taken from the collected signals. In some embodiments, the aggregation algorithm 206 collects test kinematic data measured during a therapy session. In some embodiments, the aggregation algorithm 206 collects passive kinematic data measured at times outside the therapy session.

In some embodiments, the aggregation algorithm 206 associates certain portions of the passive kinematic data with the test kinematic data. For example, In some embodiments, the aggregation algorithm 206 associates the passive kinematic data measured after a therapy session with the kinematic data measured during the therapy session. The aggregation algorithm 206 can collect the passive kinematic data after each session of a plurality therapy sessions to create a database. In this way, In some embodiments, the controller 200 can determine a level of satisfaction from the therapy session at least in part based on the passive kinematic data.

In some embodiments, the controller 200 determines a level of patient satisfaction based on the passive kinematic data without requiring the patient to input a subjective satisfaction level. In some embodiments, the aggregation algorithm 206 collects kinematic data measured during the therapy session along with satisfaction data input by the patient. In this way In some embodiments, the controller 200 can determine a level of patient satisfaction based on both the passive kinematic data and the patient provided subjective satisfaction level.

In some embodiments, the aggregation algorithm 206 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics. Data can be collected from any number of neurostimulation devices 100. The neurostimulation devices 100 can be distributed across different geographic locations, used by different patients, and configured to sense one or more of a variety of different kinematic metrics. Different sensors may be configured to monitor the same kinematic metric with varying degrees of accuracy. Each different sensor included in the neurostimulation device 100 can be individually configured to sense designated physical metrics in accordance with user or administrator instructions. Each neurostimulation device 100 can also be configured to provide a data stream of one or more sensed kinematic metrics to the aggregation algorithm 206 in accordance with user or administrator instructions. The aggregation algorithm 206 can be associated with one or more of the neurostimulation device 100, the user interface device 150, the base station 120, and/or the cloud 122.

The controller 200 can further include an aggregation learning algorithm 208. The aggregation learning algorithm 208 can determine parameters of one or more response profiles based on the aggregated data set. The aggregation learning algorithm 208 can evaluate the extracted features (e.g., for both treatment metrics and/or kinematic metrics) based on geospatial data, temporal data, disease, patient attributes or characteristics, and neurostimulation device 100 characteristics associated with the extracted features. For example, extracted features sensed at devices 100 with increased accuracy can be given more (e.g., statistical) weight relative to extracted features sensed with reduced accuracy such as by a user interface device 150. Similarly, extracted features can be weighted (e.g., statistically) based on, for example, geospatial data and/or temporal data indicating more or less closeness to a location and/or time specified in a health related determination. The aggregation learning algorithm 208 can use the aggregated features collected by the aggregation algorithm 206 to determine the one or more response profiles that correspond to one or more different neurostimulation therapies. The aggregation learning algorithm 208 can be associated with one or more of the neurostimulation device 100, the user interface device 150, the base station 120, and/or the cloud 122.

The controller 200 can further include a learning algorithm 210. In some embodiments, the aggregation learning algorithm 208 works with the learning algorithm 210 to select and personalize a response profile for an individual patient. In some embodiments, the steps described being performed by the aggregation learning algorithm 208 can instead be performed by the learning algorithm 210 or vice versa. Thus, even though the controller 200 is described as comprising both the aggregation learning algorithm 208 and the learning algorithm 210, the functionality of the aggregation learning algorithm 208 and the learning algorithm 210 can be performed by only one of the aggregation learning algorithm 208 or the learning algorithm 210. In some embodiments, implementation of the aggregation learning algorithm 208 is performed in the cloud 122 while the learning algorithm 210 is implemented in the neurostimulation device 100, the base station 120, and/or the user interface device 150.

In some embodiments, the learning algorithm 210 can select a plurality of response profiles that closely relate to the signal data collected by the controller 200 for the patient. In some embodiments, the plurality of response profiles accessed by the learning algorithm 210 can be a subset of all of the response profiles determined by the aggregation learning algorithm 208. For example, In some embodiments, the learning algorithm 210 selects the response profile(s) for which a positive outcome is predicted by the aggregation learning algorithm 208. In some embodiments, the learning algorithm 210 modifies the one or more parameters of the selected response profile based on the individual patient to further personalize the response profile.

The aggregation learning algorithm 208 and/or the learning algorithm 210 can automatically determine a correlation between specific extracted features and neurostimulation therapy outcomes. Outcomes can include, for example, identifying patients who will respond to the therapy (e.g., during an initial trial fitting or calibration process) based on tremor features of kinematic data (e.g., approximate entropy), predicting stimulation settings for a given patient (based on their tremor features) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient tremor severity at a given point, predicting patient response over time, examining patient medication responsiveness combined with tremor severity over time, predicting response to transcutaneous or percutaneous stimulation, or implantable deep brain stimulation or thalamotomy based off of tremor features and severity over time, and predicting ideal time for a patient to receive transcutaneous or percutaneous stimulation, or deep brain stimulation or thalamotomy based off of tremor features and severity over time, predicting patient reported therapy outcomes or patient reported satisfaction using tremor features assessed kinematic measurements from the device; predicting patient response to undesirable user experience using tremor features assessed from kinematic measurements and patient usage logs from the device where undesirable user experiences can include but are not limited to device malfunctions and adverse events such as skin irritation or burn; predict patient response trends based on tremor severity where trends can be assessed across total number of sessions, within an individual patient, or across a population of patients; predicting or classifying subtypes of tremor to predicting patient response based on kinematic analysis of tremor features; predicting or classifying subtypes of tremor to provide guidance for individually optimized therapy parameters; predicting or classifying subtypes of tremor to optimize the future study design based on subtypes (e.g., selecting specific subtypes of essential tremor for a clinical study with specific design addressing therapy need for the subtype); and predict patient or customer satisfaction (e.g., net promoter score) based on patient response or other kinematic features from measure tremor motion. In some embodiments, essential tremor pathology can include, for example, a primarily cerebellar variant with Bergmann gliosis and Purkinje cell torpedoes, and a Lewy body variant, and a dystonic variant, and a multiple sclerosis variant, and a Parkinson disease variant.

In some embodiments, the neuromodulation, e.g., neurostimulation device 100 or the user interface device 150 with sensors can collect kinematic or motion data, or data from other sensors, when a tremor inducing task is being performed. The patient can be directly instructed to perform the task, for example via the display on the device or audio. In some embodiments, features of tremor inducing tasks are stored on the device and used to automatically activate sensors to measure and store data to memory during relevant tremor tasks. The period of time for measuring and storing data can be, for example, 10, 20, 30, 60, 90, 120 seconds, or 1, 2, 3, 5, 10, 15, 20, 30 minutes, or 1, 2, 3, 4, 5, 6, 7, 8 hours or more or less, or ranges incorporating any two of the foregoing values. Based on a training set of data from a cohort of previous wearers with tremor or another condition, the aggregation learning algorithm 208 and/or the learning algorithm 210 can detect features that are correlated with response to stimulation such that the patient or physician can be presented with one or more response profiles. The one or more response profiles can correspond to neurostimulation therapy that has a qualitative likelihood for patient response. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

In another embodiment, features can be correlated with the type of tremor measured, such as resting tremor (associated with Parkinson's Disease), postural tremor, action tremor, intention tremor, rhythmic tremor (e.g., a single dominant frequency) or mixed tremor (e.g., multiple frequencies). The type of tremor most likely detected can be presented to the patient or physician as a diagnosis or informative assessment prior to receiving stimulation or to assess appropriateness of prescribing a neuromodulation, e.g., stimulation treatment. In another embodiment, various response profiles may be applied based on the tremor type determined; different profiles could include changes in stimulation parameters, such as frequency, pulse width, amplitude, burst frequency, duration of stimulation, or time of day stimulation is applied. In one embodiment, the user interface device 150 can include an app that asks the patient to take a self-photograph, which has the patient perform a task that has both posture and intention actions.

In some embodiments, the neuromodulation, e.g., neurostimulation device 100 can apply transcutaneous stimulation to a patient with tremor that is a candidate for implantable deep brain stimulation or thalamotomy. Tremor features and other sensor measurements of tremor severity will be used to assess response over a prespecified usage period, which could be 1 month or 3 months, or 5, 7, 14, 30, 60, or 90 days or more or less. The response to transcutaneous stimulation as assessed, for example, by the aggregation learning algorithm 208 and/or the learning algorithm 210 described herein using sensor measurements from the device can advantageously provide an assessment of the patient's likelihood to respond to implantable deep brain stimulation or other implantable or non-implantable therapies.

In some embodiments, the aggregation learning algorithm 208 and/or the learning algorithm 210 develop rules between features and one or more parameters of one or more response profiles that correspond to neurostimulation therapy outcomes. The aggregation learning algorithm 208 and/or the learning algorithm 210 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the controller 200 can use the rules developed between features and one or more parameters to automatically determine response profiles that correspond to neurostimulation therapy outcomes. The controller 200 can also use the one or more response profiles to control or change settings of the neurostimulation device, including but not limited to stimulation parameters (e.g., stimulation amplitude, frequency, patterned (e.g., burst stimulation), intervals, time of day, individual session or cumulative on time, and the like).

Accordingly, the one or more response profiles that correspond to neurostimulation therapy can improve operation of the neuromodulation, e.g., neurostimulation device, and advantageously and accurately identify potential candidates for therapy and well as various disease state and therapy parameters over time.

The neuromodulation devices, e.g., neurostimulation devices, described herein, in several embodiments, can be used for the treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). The devices described herein may also be used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. The devices described herein can be used for the treatment of inflammatory skin conditions in some embodiments. The neuromodulation devices, e.g., neurostimulation devices, described herein can be used for the treatment of chronic fatigue syndrome. The devices described herein can be used for the treatment of chronic inflammatory symptoms and flare ups. Bradykinesia, dyskinesia, rigidity may also be treated according to several embodiments. In several embodiments, rehabilitation as a result of certain events is treated, for example, rehabilitation from stroke or other cardiovascular events. In several embodiments, treatment of involuntary and/or repetitive movements is provided, including but not limited to tics, twitches, etc. (including, for example, Tourette Syndrome, tic disorders). Rhythmic and non-rhythmic involuntary movements may be controlled in several embodiments. Involuntary vocal tics and other vocalizations may also be treated. Systems and methods to reduce habituation and/or tolerance to stimulation in the disorders and symptoms identified herein are provided in several embodiments by, for example, introducing variability in stimulation parameter(s) described herein.

In several embodiments, the neuromodulation, e.g., neurostimulation, devices described herein can be used for the treatment of cardiac conditions (such as atrial fibrillation) and for the treatment of immune dysfunction.

In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s). The devices described herein can be used to stimulate the autonomic nervous system. The devices described herein can be used to balance the sympathetic/parasympathetic nervous systems. Dysfunction or imbalance of the autonomic nervous system is believed to be a potential underlying mechanism for various chronic diseases. Autonomic dysfunction develops when the nerves of the ANS are damaged or degraded. This condition is called autonomic neuropathy or dysautonomia. Autonomic dysfunction can range from mild to life-threatening and can affect part of the ANS or the entire ANS. Sometimes the conditions that cause problems are temporary and reversible. Others are chronic, or long term, and may continue to worsen over time. Examples of chronic diseases that are associated with autonomic dysfunction include, but are not limited to, diabetes, Parkinson's disease, tremor, cardiac arrhythmias including atrial fibrillation, hypertension, overactive bladder, urinary incontinence, fecal incontinence, inflammatory bowel diseases, rheumatoid arthritis, migraine, depression, and anxiety.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc. Other disorders (e.g., hypertension, dexterity, and cardiac dysrhythmias) can also be treated using the embodiments described herein.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, greater auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

The generated one or more response profiles that correspond to neurostimulation therapy can be saved in the memory 110 and/or memory 154. For example, the one or more response profiles can be generated and stored prior to operation of the neurostimulation device 100. Accordingly, in some embodiments, the controller 200 can apply the saved one or more profiles based on new data collected by the one or more sensors 112 (e.g., IMU) to determine outcomes or control the neuromodulation, e.g., neurostimulation device 100.

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

### Specific Examples of Personalized Therapy

In some embodiments, systems employing the neuromodulation device 100 utilize one or more of a decision tree, the learning algorithm 210, and/or the aggregation learning algorithm 208 to provide personalized therapy. In some embodiments employing the decision tree, the decision tree can be predetermined for a plurality of patients. In some embodiments, the patient satisfaction data (e.g., 1-4 scale) would determine the therapy path through the decision tree for the patient. In some embodiments, the decision tree is used during a trial period resulting in a coarse personalized therapy. After the trial period concludes, the final parameters associated with the coarse personalized therapy can be uploaded to the aggregation learning algorithm 208. The aggregation learning algorithm 208 and/or the learning algorithm 210 can analyze the final parameters and provide a more refined personalized therapy based on population profiles. In some embodiments, the parameters of the refined personalized therapy profiles are fed back into the predetermined decision tree to update the decision tree for future patients.

In some embodiments, the learning algorithm 210 assesses therapy kinematics and the patient satisfaction data to select the next therapy parameters during the trial period. In some embodiments at the end of iteration during the trial period, the learning algorithm 210 will have refined the therapy parameters to provide personalized therapy to the patient.

In some embodiments, the learning algorithm 210 works with the aggregation learning algorithm 208 to assess population data. In some embodiments, the aggregation learning algorithm 208 creates one or more response profiles or population ET profiles. In some embodiments, the one or more response profiles provide a starting point for either the decision tree or the learning algorithm 210. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

In some embodiments, the neuromodulation device 100 can include the ability to track a user's motion data for the purpose of gauging one, two, or more tremor frequencies of a patient. The patient could have a single tremor frequency, or in some cases multiple discrete tremor frequencies that manifest when performing different tasks. Once the tremor frequencies are observed, they can be used as one of many seminal input parameters to a personalized neuromodulation therapy. The therapy can be delivered, e.g., transcutaneously, via one, two, or more nerves (e.g., the median and radial nerves, and/or other nerves disclosed elsewhere herein) in order to reduce or improve a condition of the patient, including but not limited to their tremor burden. In some embodiments, the therapy modulates afferent nerves, but not efferent nerves. In some embodiments, the therapy preferentially modulates afferent nerves. In some embodiments, the therapy does not involve functional electrical stimulation. Although transcutaneous delivery is used in many embodiments, in some embodiments at least a portion of the devices may be implanted subcutaneously or percutaneously. In one embodiment, a first electrode stimulates the median nerve, a second electrode stimulates the radial nerve, and a third electrode stimulates the ulnar nerve. In one embodiment, two or more electrodes stimulate the same nerve (e.g., with different frequencies or other parameters). In one embodiment, one two or all of the median nerve, radial nerve, and ulnar nerve are stimulated.

In some embodiments, a hardware processor can be configured to perform any number of the following: obtain raw motion data by turning on the IMU transducer, e.g., the accelerometer; collect patient data over a selected time period (e.g., 10 seconds) (x/y/z axes); Perform a fast Fourier transform (FFT) on x-axis (yielding [symbol] (x)); perform FFT on y-axis (yielding [symbol] (y)); and/or perform FFT on z-axis (yielding [symbol] (z); derive the Total Frequency Domain (TFD)=Σ{ [symbol] (x), [symbol] (y), [symbol] (z)}; and determine the calibration frequency as the highest value of TFD (e.g., between about 2-15 Hz, 4-12 Hz, 6-10 Hz, and overlapping ranges therein in some cases).

FIG. 3 illustrates a flow chart of an embodiment of a process 300 for personalization of a neurostimulation device 100. The process 300 can be implemented by any of the systems discussed above. The process 300 can be implemented to personalize a specific neuromodulation, e.g., neurostimulation device 100 for a particular user or multiple neurostimulation devices across multiple users.

As explained above, the neurostimulation device 100 provides personalized therapy. In some embodiments, the neurostimulation device 100 provides personalization by adjusting one or more therapy parameters, such as a waveform, using a patient's or individual's tremor frequency. In some embodiments, the neurostimulation device 100 stores kinematic data and satisfaction data per session in the memory 110. In some embodiments, the aggregation algorithm 206 aggregates this data. In some embodiments, the controller 200 uses the aggregated data to reinforce the learning methods disclosed herein. For example, In some embodiments, the learning methods tune or optimize the therapy parameters, thereby increasing outcomes. In some embodiments, the controller 200 classifies the patient into a response profile. In some embodiments, the response profile provides personalized therapy parameters not only based on the patient's response but what is also known to benefit other patients with similar response profiles. Relying on data from what is also known to benefit other patients can provide a more successful outcome especially since little may be known about the underlying etiology of the patient's tremor.

In an embodiment, the process 300 begins at block 302 when the neuromodulation, e.g., neurostimulation device 100 is activated. The device can be activated in response to a user input. User input can be received via a push button or any other user interface such as the display 106 of the neuromodulation, e.g., neurostimulation device. In some embodiments, the neuromodulation, e.g., neurostimulation device 100 can be activated based on a signal received from the user interface device 150 or another computing system.

Following activation, the controller 200 can begin collecting sensor data from the sensors 112 (e.g., IMU) as well as satisfaction data. In an embodiment, the controller 200 can continue collecting sensor data for a period of 10 seconds, or about, at least about, or no more than about 1, 2, 3, 4, 5, 6, 7, 8, 9, 10, 11, 12, 13, 14, 15, 20, 25, 30, 40, 50, 60 seconds, ranges including any two of the aforementioned values, or other time periods. In some embodiments, the controller 200 can continue collecting data for longer periods of time. In some embodiments, the controller 200 collects satisfaction data from the patient during and/or after the therapy session.

The controller 200 can activate the electrodes 104 to apply neurostimulation at block 306 based on a personalized response profile that correspond to a neurostimulation therapy as described herein. The controller 200 can acquire additional data from the sensor 112 (e.g., IMU) as well as satisfaction data from the patient after the application of neurostimulation for a time period at block 308.

At block 310, the controller 200 can process the collected sensor data, including the satisfaction data, after stimulation to further determine or change one or more parameters of the personalized response profile.

In some instances, the personalization can be processing and/or data intensive. Particularly, when the personalization is performed on the neurostimulation device 100, the onboard hardware processors and memory may not have enough processing or memory capabilities to process the features extracted by the feature extraction engine 204 to determine one or more response profiles that correspond to one or more different neurostimulation therapies. To reduce the memory usage, it may be advantageous in some instances for one or more of the aggregation algorithm 206, the aggregation learning algorithm 208, and/or the learning algorithm 210 to be remotely located relative to the neurostimulation device 100. Accordingly, in some instances, the aggregation algorithm 206 and/or the aggregation learning algorithm 208 are located in the cloud 122. In some other instances, the aggregation algorithm 206 and/or the aggregation learning algorithm 208 are located in the neurostimulation device 100, the user interface device 150, and/or the base station 120. In some instances, the learning algorithm 210 is located in the cloud 122. In some other instances, the learning algorithm 210 is located in the neurostimulation device 100, the user interface device 150, and/or the base station 120.

In some embodiments, the aggregation learning algorithm 208 and/or the learning algorithm 210 employ machine learning algorithms including reinforcement learning (RL) to 1) optimize therapy parameters based on individual response/satisfaction and/or 2) assign a therapy response profile to influence further therapy titration.

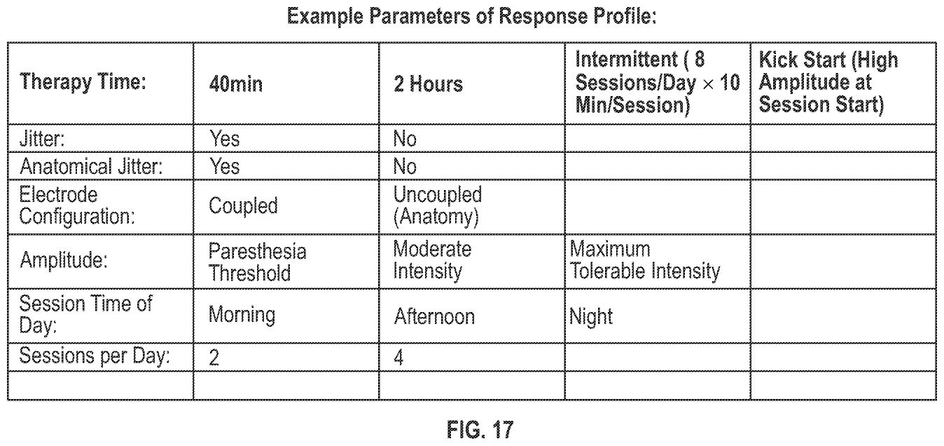

The aggregation learning algorithm 208 and/or the learning algorithm 210 can add additional parameters and parameter options as new ones arise. In some embodiments, therapy parameters are listed in FIG. 17. Additional data sources, such as the user interface device 150, may strengthen the machine learning model. For example, the additional data sources can provide data/feedback gathered from subjective patient feedback and objective patient-specific data. The additional data sources can provide medical information and/or medical records specific to a patient. In some embodiments, the data is sent to the cloud 122 or communicated directly to the neurostimulation device 100. In some embodiments, the data can be used as a trigger to start/stop therapy.

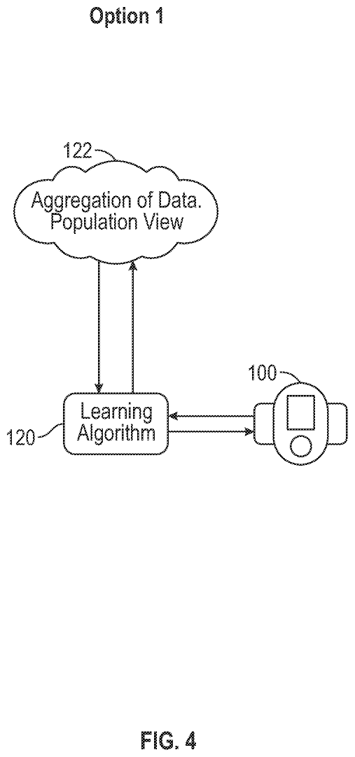

FIG. 4 schematically illustrates an embodiment of a system for personalization of the neurostimulation device 100 employing the learning algorithm 210 in the base station 120. In some embodiments, the learning algorithm 210 leverages patient satisfaction data and kinematic data downloaded nightly from the neurostimulation device 100 to the base station 120. In some embodiments, the therapy parameters initially provided in the response profile from the aggregation learning algorithm 208 are updated by the learning algorithm 210 to provide a more refined therapeutic session for the patient that are predicted to improve outcomes. In some embodiments, the therapy parameters provided by the aggregation learning algorithm 208 are updated each new day or every few days.

Locating the learning algorithm 210 in the base station 120 may provide certain advantages for the controller 200. For example, when the learning algorithm 210 is located in the base station 120, the neurostimulation device 100 and the base station 120 can share data when the neurostimulation device 100 is docked to the base station 120 via a wired or direct data connection. In some embodiments, the base station 120 and the neurostimulation device 100 share data when the neurostimulation device 100 is being charged by the base station 120. In other embodiments, the base station 120 and the neurostimulation device 100 wirelessly transmit and receive data. With the learning algorithm 210 located in the base station 120, the controller 200 can consistently update the neurostimulation device 100 to provide a more tailored therapeutic experience to the patient. In the illustrated embodiment, the aggregation algorithm 206 and the aggregation learning algorithm 208 are located in the cloud 122.

In some embodiments, the aggregation algorithm 206 collects the extracted features taken from the collected signals. In some embodiments, the aggregation algorithm 206 collects test kinematic data measured during a therapy session. In some embodiments, the aggregation algorithm 206 collects passive kinematic data measured at times outside the therapy session. In some embodiments, the aggregation algorithm 206 collects kinematic data measured during the therapy session along with satisfaction data input by the patient. In some embodiments, the aggregation algorithm 206 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

The aggregation learning algorithm 208 can use the aggregated features collected by the aggregation algorithm 206 to determine the one or more response profiles that correspond to one or more different neurostimulation therapies. In the illustrated embodiment, the learning algorithm 210 transmits and receives data with the aggregation learning algorithm 208.

In some embodiments, the learning algorithm 210 leverages satisfaction data (e.g., 1-4 scale) and kinematic data downloaded (e.g., at certain time intervals, such as nightly) from the neurostimulation device 100 to the base station 120.

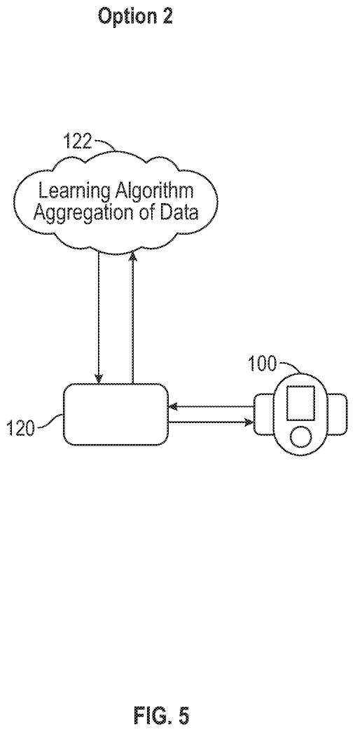

FIG. 5 schematically illustrates an embodiment of a system for personalization of the neurostimulation device 100 employing the learning algorithm 210 in the cloud 122. In some embodiments, the learning algorithm 210 leverages patient satisfaction data and kinematic data from the neurostimulation device 100. The neurostimulation device 100 provides the data to the base station 120 which then uploads the data to the cloud 122. In some embodiments, the therapy parameters initially provided in the response profile from the aggregation learning algorithm 208 are updated by the learning algorithm 210 to provide a more refined therapeutic session for the patient that are predicted to improve outcomes. In some embodiments, the therapy parameters provided by the aggregation learning algorithm 208 are updated each new day or every few days (e.g., every 12, 24, 36, 48, 72 hours etc.).

Locating the learning algorithm 210 in the cloud 122 may provide certain advantages for the controller 200. For example, when the learning algorithm 210 is located in the cloud 122, the learning algorithm 210 can leverage third party computing resources which may exceed the computing resources available in the neurostimulation device 100 and/or the base station 120. While the neurostimulation device 100 and the base station 120 can share data when the neurostimulation device 100 is docked to the base station 120, the availability of the one or more response profiles will also depend on the sharing of data between the base station 120 and the cloud 122. As compared to the system illustrated in FIG. 4 in which the learning algorithm 210 is local to the neurostimulation device 100, the quality of the data connection between the cloud 122 and the base station 120 in FIG. 5 may vary depending on, for example, network and environmental factors.

In some embodiments, the base station 120, the neurostimulation device 100, and the cloud 122 share data when the neurostimulation device 100 is being charged by the base station 120. For example, in addition to the connection between the base station 120 and the neurostimulation device 100, the base station 120 is further connected, wired or wirelessly, to a local network. In other embodiments, the base station 120 and the neurostimulation device 100 wirelessly transmit and receive data. In the illustrated embodiment, the aggregation algorithm 206 and the aggregation learning algorithm 208 are located in the cloud 122.

In some embodiments, the aggregation algorithm 206 collects the extracted features taken from the collected signals. In some embodiments, the aggregation algorithm 206 collects test kinematic data measured during a therapy session. In some embodiments, the aggregation algorithm 206 collects passive kinematic data measured at times outside the therapy session. In some embodiments, the aggregation algorithm 206 collects kinematic data measured during the therapy session along with satisfaction data input by the patient. In some embodiments, the aggregation algorithm 206 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

The aggregation learning algorithm 208 can use the aggregated features collected by the aggregation algorithm 206 to determine the one or more response profiles that correspond to one or more different neurostimulation therapies. In the illustrated embodiment, the learning algorithm 210 transmits and receives data with the base station 120.

In some embodiments, a predefined decision tree is utilized to personalize therapy. For example, In some embodiments, a predefined decision tree is embedded in the neurostimulation device 100. The neurostimulation device 100 can leverage the predefined decision tree and satisfaction data to coarsely refine therapy parameters. In some embodiments, the neurostimulation device 100 coarsely refines the therapy parameters session to session or after multiple sessions depending on the predetermined decision tree.

In some embodiments, the learning algorithm 210 is processed in the cloud 122. In some embodiments, the learning algorithm 122 can leverage satisfaction data (1-4 scale) and kinematic data downloaded nightly from the neurostimulation device 100 to the base station 120, and from the base station 120 to the cloud 122. With each new day or every few days, the learning algorithm 122 updates the therapy parameters to provide a more refined therapeutic session for the patient that are predicted to improve outcomes.

In some embodiments, the aggregation learning algorithm 208 leverages populations of data to create different therapy profiles based on demographics and disease history. In some embodiments, the learning algorithm 210 works with a predetermined decision tree. In some embodiments, the predetermined decision tree is embedded in the neurostimulation device 100. In some embodiments, the learning algorithm 210 selects a specific predetermined decision tree based on the patient's ET profile created by the aggregation learning algorithm 208.

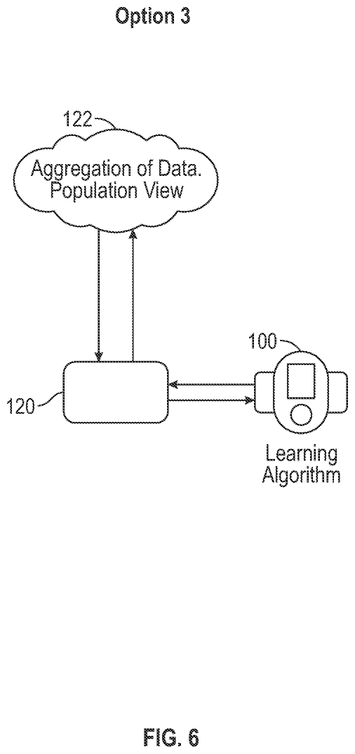

FIG. 6 schematically illustrates an embodiment of a system for personalization of the neurostimulation device 100 employing the learning algorithm 210 in the neuromodulation device 100. In some embodiments, the learning algorithm 210 leverages patient satisfaction data and kinematic data from the neurostimulation device 100. In some embodiments, the neurostimulation device 100 provides the data to the base station 120 which then uploads the data to the cloud 122. In some embodiments, the therapy parameters initially provided in the response profile from the aggregation learning algorithm 208 are updated by the learning algorithm 210 to provide a more refined therapeutic session for the patient that are predicted to improve outcomes. In some embodiments, the therapy parameters provided by the aggregation learning algorithm 208 are updated each new day or every few days.

Locating the learning algorithm 210 in the neurostimulation device 100 may provide certain advantages for the controller 200. For example, when the learning algorithm 210 is located in the neurostimulation device 100, the learning algorithm 210 can update the response profile received from the aggregation learning algorithm 208 in real time. In some embodiments, the base station 120 need only periodically access the cloud 122 to receive a response profile. In this way, the response profile from the cloud 122 can be a base line response profile. The neurostimulation device 100 can subsequently update the response profile each day, multiple times a day, or after every therapy session without having to dock with the base station 120 or rely on a data connection between the base station 120 and the cloud 122 to further personalize the response profile. In the illustrated embodiment, the aggregation algorithm 206 and the aggregation learning algorithm 208 are located in the cloud 122.

In some embodiments, the aggregation algorithm 206 collects the extracted features taken from the collected signals. In some embodiments, the aggregation algorithm 206 collects test kinematic data measured during a therapy session. In some embodiments, the aggregation algorithm 206 collects passive kinematic data measured at times outside the therapy session. In some embodiments, the aggregation algorithm 206 collects kinematic data measured during the therapy session along with satisfaction data input by the patient. In some embodiments, the aggregation algorithm 206 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

The aggregation learning algorithm 208 can use the aggregated features collected by the aggregation algorithm 206 to determine the one or more response profiles that correspond to one or more different neurostimulation therapies. In the illustrated embodiment, the learning algorithm 210 transmits and receives data with the base station 120.

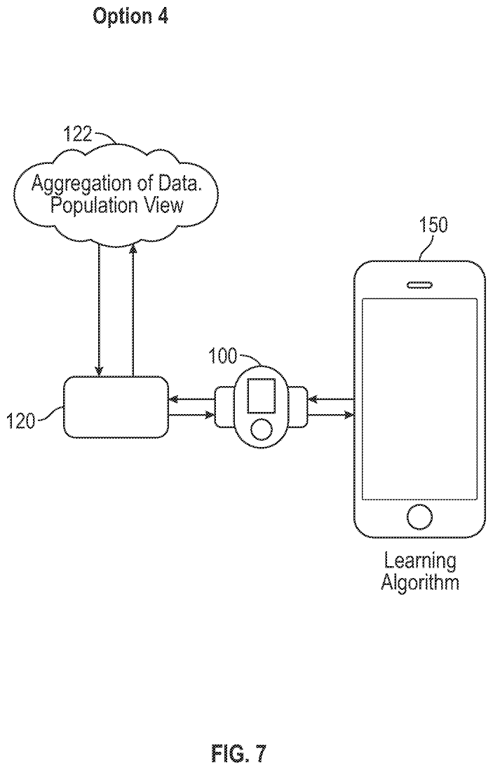

FIG. 7 schematically illustrates an embodiment of a system for personalization of the neurostimulation device 100 employing the learning algorithm 210 in a third party device such as, for example, the user interface device 150. In some embodiments, the learning algorithm 210 leverages patient satisfaction data and kinematic data received from the neurostimulation device 100. The neurostimulation device 100 provides the data to the base station 120 which then uploads the data to the cloud 122. In some embodiments, the therapy parameters initially provided in the response profile from the aggregation learning algorithm 208 are updated by the learning algorithm 210 to provide a more refined therapeutic session for the patient that are predicted to improve outcomes. The updated therapy parameters are provided by the user interface device 150 to the neurostimulation device 100. In some embodiments, the therapy parameters provided by the aggregation learning algorithm 208 are updated each new day or every few days.

Locating the learning algorithm 210 in the user interface device 150 may provide certain advantages for the controller 200. For example, when the learning algorithm 210 is located in the user interface device 150, the learning algorithm 210 can leverage third party computing resources which may exceed the computing resources available in the neurostimulation device 100 and/or the base station 120. When the learning algorithm 210 is located in the user interface device 150, the learning algorithm 210 can leverage medical data or records received by the user interface device 150. While the neurostimulation device 100 and the base station 120 can share data when the neurostimulation device 100 is docked to the base station 120, the availability of the one or more response profiles will also depend on the sharing of data between the neurostimulation device 100 and the user interface device 150. As compared to the system illustrated in FIG. 4 in which the learning algorithm 210 is local to the neurostimulation device 100, the quality of the data connection between the user interface device 150 and the neurostimulation device 100 in FIG. 7 may vary depending on, for example, network and environmental factors.

In some embodiments, the user interface device 150 and the neurostimulation device 100 wirelessly transmit and receive data. In the illustrated embodiment, the aggregation algorithm 206 and the aggregation learning algorithm 208 are located in the cloud 122.

In some embodiments, the aggregation algorithm 206 collects the extracted features taken from the collected signals. In some embodiments, the aggregation algorithm 206 collects test kinematic data measured during a therapy session. In some embodiments, the aggregation algorithm 206 collects passive kinematic data measured at times outside the therapy session. In some embodiments, the aggregation algorithm 206 collects kinematic data measured during the therapy session along with satisfaction data input by the patient. In some embodiments, the aggregation algorithm 206 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics.

The aggregation learning algorithm 208 can use the aggregated features collected by the aggregation algorithm 206 to determine the one or more response profiles that correspond to one or more different neurostimulation therapies. In the illustrated embodiment, the learning algorithm 210 transmits and receives data with the neurostimulation device 100.

In certain embodiment, the learning algorithm 210 is processed in the user interface device 150. The learning algorithm 210 can leverages satisfaction data (1-4 scale) and kinematic data downloaded session to session from the neurostimulation device 100 to the user interface device 150. In some embodiments, with each new session or after multiple sessions, the learning algorithm 210 updates the therapy parameters to provide a more refined therapeutic session for the patient that are predicted to improve outcomes.

### Aggregate Satisfaction Data

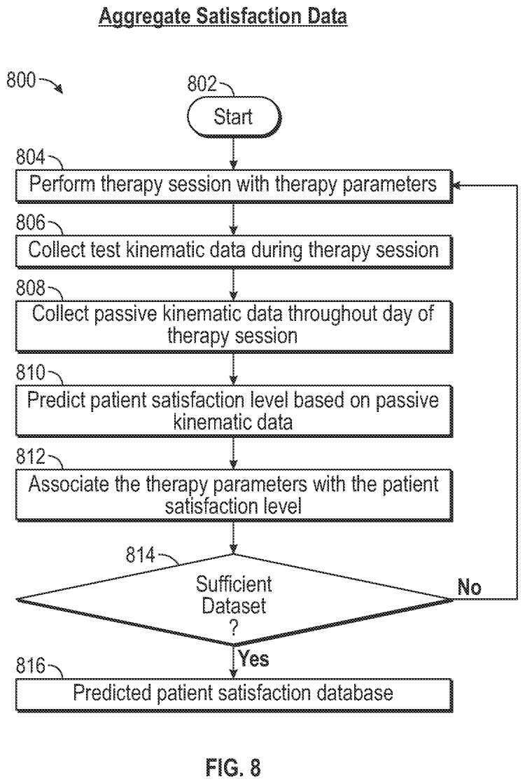

FIG. 8 illustrates a flow chart of a non-limiting embodiment of a process 800 for aggregating passive satisfaction data. The process 800 can be implemented by any of the systems described above. In some embodiments, subjective patient scoring of therapy benefit is not easily correlated with objective kinematic data showing increase or decrease in tremor severity. In certain cases, even when the patient tremor is reduced, the patient may not consider the reducing in tremor severity to have meaningfully improved their life. Thus, as reduction in tremor severity may not correspond with an improvement in what is meaningful to the individual patient, such as ADL's like writing, dialing a phone, typing, or eating/drinking. In such cases, the learning algorithm 210 can provide smarter therapy settings and more automatic optimization by adjusting therapy parameters to outcomes meaningful to the patient and not just to objective data measures.

The method below leverages patient activity data provided by the sensors 112 (e.g., IMU). In some embodiments, relying on kinematic data from the sensors 112 without polling the patient to input satisfaction scores can be less burdensome to the patient. While the method can optionally employ polling the patient, In some embodiments, the patient need not input their activity at time of data capture. Without patient input, the data provided by the sensors 112 is unrelated to a specific activity and results on the data being considered noisy. If the aggregation learning algorithm 208 and/or learning algorithm 210 labels the noisy kinematic data sets with patient satisfaction for a large enough set, then future kinematic data sets captured by the sensors 112 can be used to predict patient satisfaction with tremor level and tremor improvements during activity. In some embodiments, future therapy optimization can be tuned to what is predicted to improve patient satisfaction rather than strictly based on a comparison between raw sensors 112 data.

In some embodiments, the process begins at block 802 and then moves to block 804 where the controller 200 performs a therapy session on the patient. The therapy session can employ a set of predefined therapy parameters. In some embodiments, the therapy parameters can be defined in a predetermined decision tree or based on one or more profiles. Further, block 804 can be indicative of a trial period. In some embodiments, the trial period can be defined by multiple session and/or multiple days. For example, In some embodiments, the trial period is 10 days. Of course, the trial period is not limited to 10 days and can have a length of any number of days. Further, multiple session can be conducted on a single day. In other embodiments, the interval between sessions is greater than one day.

At block 806, the controller 200 collects test kinematic data during the therapy session. For example, the controller 200 can collect test kinematic data from the sensors 112 (accelerometers, gyros) onboard the neurostimulation device 100 worn on a patient's wrist, at therapy start, during therapy, and/or upon therapy completion. The test kinematic data can include raw accelerometer and/or gyroscope data, and/or other sensor data. In an embodiment, the sensor data can include data from each of the three axes.

At block 808, the controller 200 collects passive kinematic data through the day of the therapy session. The collected passive data can be collected at various intervals throughout daily use. The passive data collection will inform how therapy impacts response duration and therefore overall satisfaction over time.

At block 810, the controller 200 predicts a patient satisfaction level based on the passive kinematic data. In some embodiments, the method can employ the aggregation learning algorithm 208 and/or learning algorithm 210 to predict the patient satisfaction level. At block 812, the controller 200 associates the therapy parameters used during the therapy session with the predicted patient satisfaction level. In some embodiments, the collected data is stored in a predicted patient satisfaction database.

At decision block 814, the controller 200 determines whether there is sufficient data in the dataset to successfully predict patient satisfaction. In some embodiments, the process returns to block 804 and repeats blocks 806-812 until there is sufficient data in the dataset. Once there is sufficient data in the dataset, the method moves to block 816 where the controller 200 publishes the predicted patient satisfaction database. In some embodiments, the predicted patient satisfaction database is provided to the neurostimulation device 100.

### Predict Therapy Satisfaction

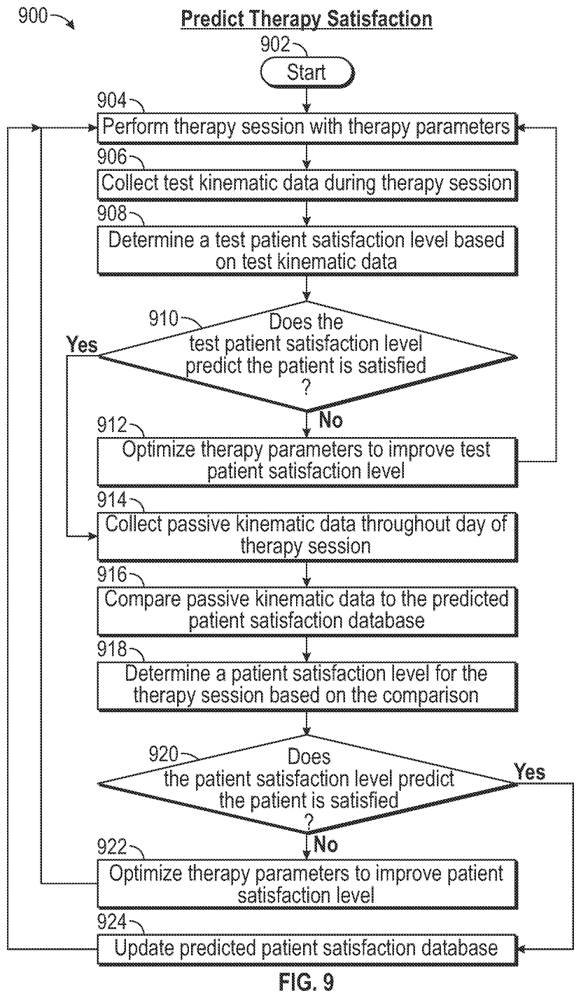

FIG. 9 illustrates a flow chart of a non-limiting embodiment of a process 900 for personalizing therapeutic protocols employing the aggregated satisfaction data from FIG. 8. The process 900 can be implemented by any of the systems described above.

In some embodiments, the process begins at block 902 and then moves to block 904 where the controller 200 performs a therapy session on the patient. The therapy session can employ a set of predefined therapy parameters. In some embodiments, the therapy parameters can be defined in a predetermined decision tree or based on one or more profiles. Further, block 904 can be indicative of a trial period. In some embodiments, the trial period can be defined by multiple session and/or multiple days. For example, In some embodiments, the trial period is 10 days. Of course, the trial period is not limited to 10 days and can have a length of any number of days. Further, multiple session can be conducted on a single day. In other embodiments, the interval between sessions is greater than one day.

At block 906 the controller 200 collects test kinematic data during the therapy session. For example, the controller 200 can collect test kinematic data from the sensors 112 (accelerometers, gyros) onboard the neurostimulation device 100 worn on a patient's wrist, at therapy start, during therapy, and/or upon therapy completion. The test kinematic data can include raw accelerometer and/or gyroscope data, and/or other sensor data. In an embodiment, the sensor data can include data from each of the three axes.

At block 908, the controller 200 determines a patient satisfaction level based on the test kinematic data. In some embodiments, the method can employ the aggregation learning algorithm 208 and/or learning algorithm 210 to determine the patient satisfaction level. The controller 200 determines the test patient satisfaction level based on objective test data. However, as explained above, improvement in the test patient satisfaction level may not be as meaningful to the patient.

At decision block 910, the controller 200 determines whether the test patient satisfaction level determined at block 908 predicts the patient is satisfied. In some embodiments, the process returns to block 904 and repeats blocks 906-908 until the controller 200 predicts the patient is satisfied based on the satisfaction level determined at block 908.

Once the controller 200 predicts the patient is satisfied, the method moves to block 914 where the controller 200 collects passive kinematic data through the day of the therapy session. The collected passive data can be collected at various intervals throughout daily use. The passive data collection will inform how therapy impacts response duration and therefore overall satisfaction over time.

At block 916, the controller 200 compares the passive kinematic data to the predicted patient satisfaction database. The predicted patient satisfaction database was determined by the process outlined in FIG. 8.

At block 918, the controller 200 determines a patient satisfaction level for the therapy session based on the comparison. In some embodiments, the controller 200 is comparing the passive kinematic data collected throughout the day to the predicted patient satisfaction database.

At block 920, the controller 200 determines whether the patient satisfaction level determined at block 918 predicts the patient is meaningfully satisfied. If the controller 200 predicts the patient is not satisfied, the method moves to block 922. At block 922, the controller 200 can employ the aggregation learning algorithm 208 and/or learning algorithm 210 to optimize the therapy parameters to improve the patient satisfaction level. The method can then repeat blocks 904-918 until the controller 200 determines the patient satisfaction level determined at block 918 predicts the patient is meaningfully satisfied.

At block 924, the therapy parameters which resulted in the patient being meaningfully satisfied can be used to update the predicted patient satisfaction database determined by the process of FIG. 8.

### Individual Data Aggregation

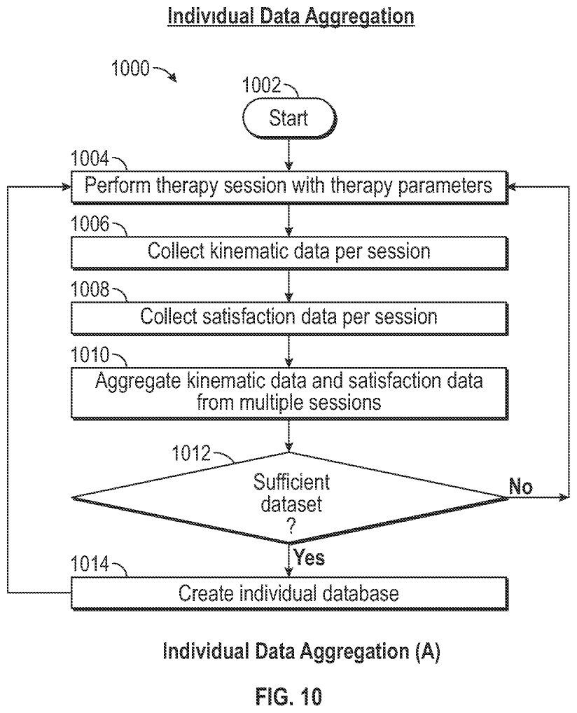

FIG. 10 illustrates a flow chart of a non-limiting embodiment of a process 1000 for aggregating data collected from an individual including kinematic and satisfaction data. The process 1000 can be implemented by any of the systems described above. The method below leverages satisfaction data to improve therapy outcomes. In some embodiments, the method relies on kinematic data from the sensors 112 along with polling the patient to input satisfaction scores. With patient input, the data provided by the sensors 112 is related to a specific activity.

In some embodiments, the process begins at block 1002 and then moves to block 1004 where the controller 200 performs a therapy session on the patient. The therapy session can employ a set of predefined therapy parameters. In some embodiments, the therapy parameters can be defined in a predetermined decision tree or based on one or more profiles. Further, block 1004 can be indicative of a trial period. In some embodiments, the trial period can be defined by multiple session and/or multiple days. For example, In some embodiments, the trial period is 10 days. Of course, the trial period is not limited to 10 days and can have a length of any number of days. Further, multiple session can be conducted on a single day. In other embodiments, the interval between sessions is greater than one day.

At block 1006, the controller 200 collects kinematic data during the therapy session. For example, the controller 200 can collect kinematic data from the sensors 112 (accelerometers, gyros) onboard the neurostimulation device 100 worn on a patient's wrist, at therapy start, during therapy, and/or upon therapy completion. The kinematic data can include raw accelerometer and/or gyroscope data, and/or other sensor data. In an embodiment, the sensor data can include data from each of the three axes.

At block 1008, the controller 200 collects satisfaction data. The satisfaction data can be collected at therapy start, during therapy, and/or upon therapy completion. The collected satisfaction data can be collected at various intervals throughout daily use. The kinematic data and the satisfaction data will inform how therapy impacts response duration and therefore overall satisfaction over time.

At block 1010, the controller 200 aggregates the collected kinematic data and the collected satisfaction data. In some embodiments, the method can employ the aggregation learning algorithm 208 and/or the learning algorithm 210 to aggregate the kinematic data and the satisfaction data.

At decision block 1012, the controller 200 determines whether there is sufficient data in the dataset to successfully predict patient satisfaction. In some embodiments, the process returns to block 1004 and repeats blocks 1006-1012 until there is sufficient data in the dataset. Once there is sufficient data in the dataset, the method moves to block 1014 where the controller 200 publishes the individual database. In some embodiments, the individual database is provided to the neurostimulation device 100.

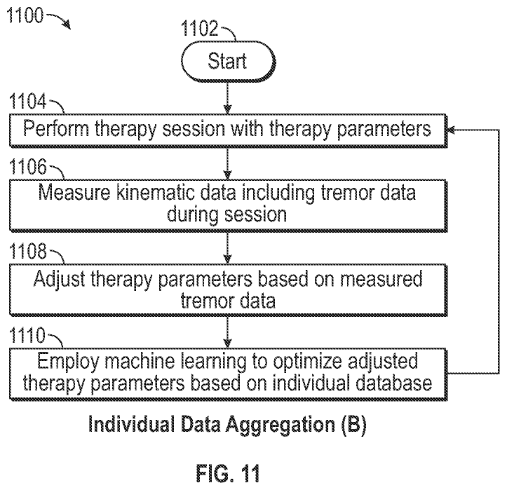

FIG. 11 illustrates a flow chart of a non-limiting embodiment of a process 1100 for personalizing therapeutic protocols employing the aggregated satisfaction data from FIG. 10. The process 1100 can be implemented by any of the systems described above.

In some embodiments, the process begins at block 1102 and then moves to block 1104 where the controller 200 performs a therapy session on the patient. The therapy session can employ a set of predefined therapy parameters. In some embodiments, the therapy parameters can be defined in a predetermined decision tree or based on one or more profiles. Further, block 1104 can be indicative of a trial period. In some embodiments, the trial period can be defined by multiple session and/or multiple days. For example, In some embodiments, the trial period is 10 days. Of course, the trial period is not limited to 10 days and can have a length of any number of days. Further, multiple session can be conducted on a single day. In other embodiments, the interval between sessions is greater than one day.

At block 1106 the controller 200 collects kinematic data during the therapy session. For example, the controller 200 can collect kinematic data from the sensors 112 (accelerometers, gyros) onboard the neurostimulation device 100 worn on a patient's wrist, at therapy start, during therapy, and/or upon therapy completion. The kinematic data can include raw accelerometer and/or gyroscope data, and/or other sensor data. In an embodiment, the sensor data can include data from each of the three axes.

At block 1108, the controller 200 can employ the aggregation learning algorithm 208 and/or learning algorithm 210 to adjust the therapy parameters based on sensor data. In some embodiments, the sensor data includes tremor data.

At block 1110, the controller 200 can employ the aggregation learning algorithm 208 and/or learning algorithm 210 to optimize the adjusted therapy parameters based on the individual database determined by the process of FIG. 10.

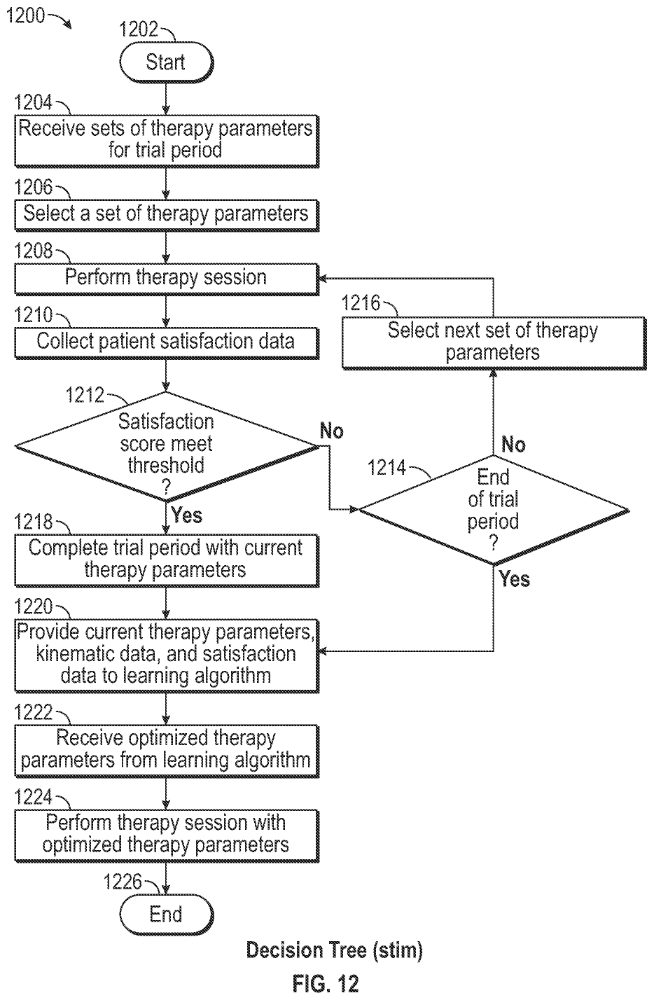

FIG. 12 illustrates a flow chart of a non-limiting embodiment of a process 1200 for personalizing therapeutic protocols employing a decision tree during a trial period and the learning algorithm 210. The process 1200 can be implemented by any of the systems described above.

The process begins at block 1202 and then moves to block 1204 where the controller 200 receives sets of therapy parameters to select from for use during a trial period. In some embodiments, the trial period can be defined by multiple session and/or multiple days. For example, In some embodiments, the trial period is 10 days. Of course, the trial period is not limited to 10 days and can have a length of any number of days. Further, multiple session can be conducted on a single day. In other embodiments, the interval between sessions is greater than one day.

At block 1206 the controller 200 selects a first set of therapy parameters from the sets of therapy parameters. In some embodiments, the therapy parameters can be defined in a predetermined decision tree or based on one or more profiles. The order of selecting each set of therapy parameters can be predetermined. In some embodiments, the controller 200 determines the order of selection based on, for example, a condition or disease state of the patient.

At block 1208, the controller 200 performs a therapy session using the selected set of therapy parameters. The therapy session can employ the selected set of therapy parameters.

At block 1210, the controller 200 collects patient satisfaction data. The satisfaction data can be collected at therapy start, during therapy, and/or upon therapy completion. The collected satisfaction data can be collected at various intervals throughout daily use. The satisfaction data will inform how therapy impacts response duration and therefore overall satisfaction over time.

At decision block 1212, the controller 200 determines whether the satisfaction score meets a predetermined threshold. Meeting the threshold indicates the patient is satisfied with the current set of therapy parameters. However, In some embodiments, meeting the threshold means there is still room for improving patient satisfaction. The threshold can be predetermined by the controller 200 or the patient. In some embodiments, the patient selects the threshold. For example, the patient can select a score of 3 on a scale from 1 to 4 as the threshold.

If the satisfaction score does not meet the threshold, the process moves to decision block 1214 where the controller 200 determines whether the trial period has ended. The length of the trial period can be based at least in part on the number of sets of therapy parameters received at block 1204. If the controller 200 determines that the trial period has not ended, the process moves to block 1216 where the controller 200 selects the next set of therapy parameters. The process then returns to block 1208 where another therapy session is performed with the newly selected set of therapy parameters.

If at decision block 1212, the controller 200 determines the satisfaction score meets the threshold, the process moves to block 1218 where the controller 200 completes the trial period with the current parameters. Alternatively, the process skips the remainder of the trial period and moves directly to block 1220.

At block 1220, the controller 200 provides the current therapy parameters, kinematic data, and satisfaction data to the learning algorithm 210. In some embodiments, the learning algorithm 210 is located in the neurostimulation device 100, the base station 120 and/or the cloud 122. Returning to decision block 1214, if the trial period has ended the process moves to block 1220 even though the satisfaction score did not meet the threshold.

At block 1222 the controller 200 receives optimized therapy parameters from the learning algorithm 210. At block 1224, the controller 200 performs a therapy session using the optimized therapy parameters received from the learning algorithm 210. The therapy session can employ the optimized therapy parameters. The process ends at block 1226.

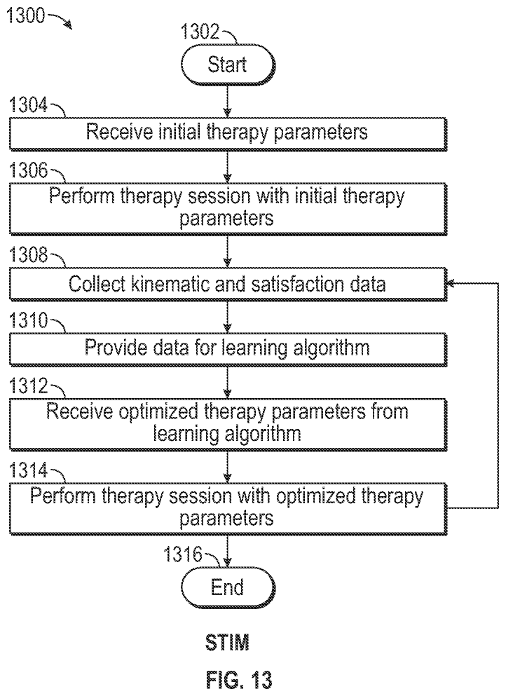

FIG. 13 illustrates a flow chart of a non-limiting embodiment of a process performed by the neurostimulation device employing the learning algorithm 210. The process 1300 can be implemented by any of the systems described above.

The process begins at block 1302 and then moves to block 1304 where the controller 200 receives initial therapy parameters. At block 1306 the controller 200 performs a therapy session using the initial therapy parameters. The therapy session can employ the initial therapy parameters.

At block 1308, the controller 200 collects kinematic data and satisfaction data from the therapy session. For example, the controller 200 can collect kinematic data from the sensors 112 (accelerometers, gyros) onboard the neurostimulation device 100 worn on a patient's wrist, at therapy start, during therapy, and/or upon therapy completion. The kinematic data can include raw accelerometer and/or gyroscope data, and/or other sensor data. In an embodiment, the sensor data can include data from each of the three axes. The satisfaction data can be collected at therapy start, during therapy, and/or upon therapy completion. The collected satisfaction data can be collected at various intervals throughout daily use. The kinematic data and the satisfaction data will inform how therapy impacts response duration and therefore overall satisfaction over time.

At block 1310, the controller 200 provides the current therapy parameters, kinematic data, and satisfaction data to the learning algorithm 210. In some embodiments, the learning algorithm 210 is located in the neurostimulation device 100, the base station 120 and/or the cloud 122.

At block 1312, the controller 200 receives optimized therapy parameters from the learning algorithm 210. At block 1314, the controller 200 performs a therapy session using the optimized therapy parameters received from the learning algorithm 210. The therapy session can employ the optimized therapy parameters. The process can return to block 1308 where the controller 200 can collect kinematic data and satisfaction data for the therapy session. The process can then repeat blocks 1310-1314 to further iterate the therapy parameters to improve outcomes of the patient. The process ends at block 1316.

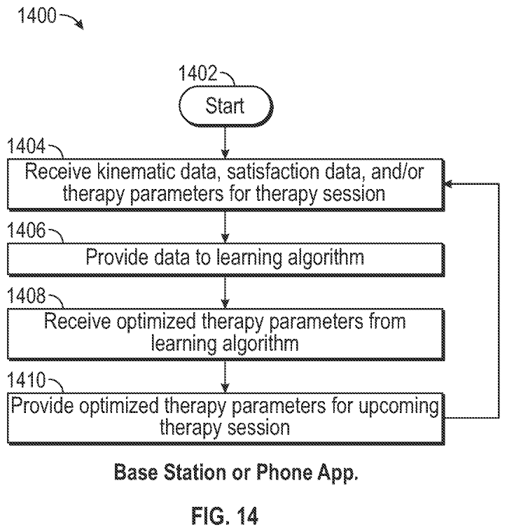

FIG. 14 illustrates a flow chart of a non-limiting embodiment of a process 1400 performed by the base station 120 or third party device such as the user interface device 150 employing the learning algorithm 210. The process 1200 can be implemented by any of the systems described above.

The process begins at block 1402 and then moves to block 1404 where the base station 120 or user interface device 150 receives the current therapy parameters, kinematic data, and satisfaction data. The process then moves to block 1406 where the controller 200 provides the current therapy parameters, kinematic data, and satisfaction data to the learning algorithm 210. In some embodiments, the learning algorithm 210 is located in the neurostimulation device 100, the base station 120, the user interface device 150, and/or the cloud 122.

At block 1408, the controller 200 receives optimized therapy parameters from the learning algorithm 210. At block 1410, the controller 200 performs a therapy session using the optimized therapy parameters received from the learning algorithm 210. The therapy session can employ the optimized therapy parameters.

The process can return to block 1404 where the controller 200 can receive kinematic data, satisfaction data, and/or therapy parameters for the therapy session. The process can then repeat blocks 1404-1410 to further iterate the therapy parameters to improve outcomes of the patient.

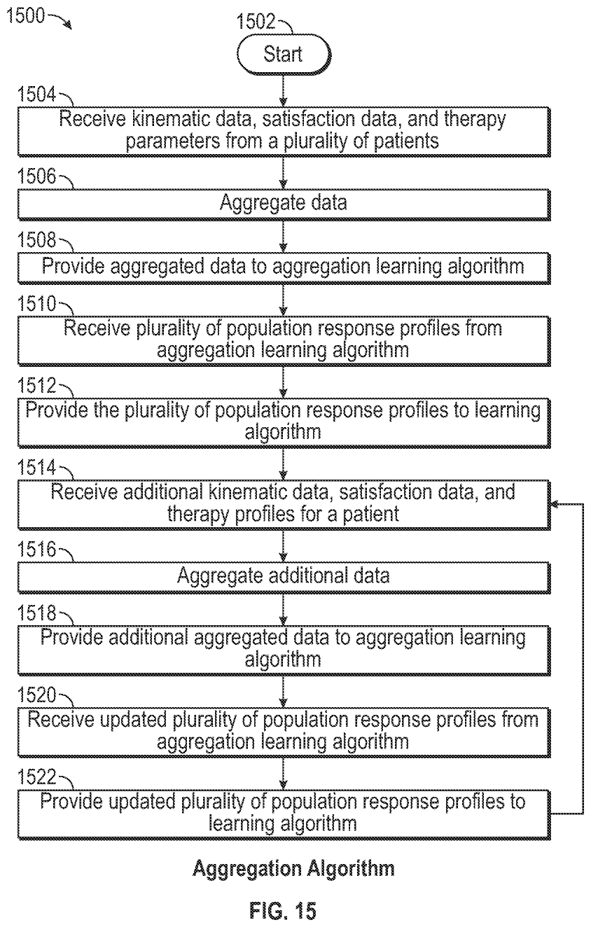

FIG. 15 illustrates a flow chart of a non-limiting embodiment of a process 1500 for aggregating data. The process 1500 can be implemented by any of the systems described above. In some embodiments, the process 1500 is implemented by the aggregation algorithm 206 of the controller 200.

The process begins at block 1502 and then moves to block 1504 where the controller 200 receives kinematic data, satisfaction data, and therapy parameters from a plurality of patients. At least some of the data can be extracted features taken from the collected signals. In some embodiments, the controller 200 collects data measured during a therapy session. In some embodiments, the controller 200 collects data measured at times outside the therapy session.

At block 1506 the controller 200 aggregates the data. In some embodiments, the controller 200 associates certain portions of passive kinematic data with test kinematic data. For example, In some embodiments, the controller 200 associates the passive kinematic data measured after a therapy session with the kinematic data measured during the therapy session. The controller 200 can collect the passive kinematic data after each session of a plurality therapy sessions to create a database. In this way, In some embodiments, the controller 200 can determine a level of satisfaction from the therapy session at least in part based on the passive kinematic data. In some embodiments, the controller 200 collects kinematic data measured during the therapy session along with satisfaction data input by the patient. In this way In some embodiments, the controller 200 can determine a level of patient satisfaction based on both the passive kinematic data and the patient provided subjective satisfaction level.

In some embodiments, the controller 200 collects data from the feature extraction engines 204 of a plurality of users or patients. In some embodiments, the aggregation or collection of data taken from multiple different patients may be taken from subpopulations of patients with like characteristics to the treated patient. In some embodiments, the subpopulations may be groups of patients with similar characteristics, e.g., age ranges, gender, hormone levels, drugs used, and/or tremor characteristics. Data can be collected from any number of neurostimulation devices 100. The neurostimulation devices 100 can be distributed across different geographic locations, used by different patients, and configured to sense one or more of a variety of different kinematic metrics. Different sensors may be configured to monitor the same kinematic metric with varying degrees of accuracy. Each different sensor included in the neurostimulation device 100 can be individually configured to sense designated physical metrics in accordance with user or administrator instructions. Each neurostimulation device 100 can also be configured to provide a data stream of one or more sensed kinematic metrics to the controller 200 in accordance with user or administrator instructions. The controller 200 can be associated with one or more of the neurostimulation device 100, the user interface device 150, the base station 120, and/or the cloud 122.

At block 1508, the controller 200 provides the aggregated data to the aggregation learning algorithm 208. The aggregation learning algorithm 208 can determine parameters of one or more response profiles from the aggregated data set. The aggregation learning algorithm 208 can evaluate the extracted features (e.g., for both treatment metrics and/or kinematic metrics) based on geospatial data, temporal data, and device 100 characteristics associated with the extracted features. The aggregation learning algorithm 208 can use the aggregated features collected by the aggregation algorithm 206 to determine the one or more response profiles that correspond to one or more different neurostimulation therapies. The aggregation learning algorithm 208 can be associated with one or more of the neurostimulation device 100, the user interface device 150, the base station 120, and/or the cloud 122.

At block 1510, the controller 200 receives a plurality of response profiles from the aggregation learning algorithm 208. At block 1512, the controller 200 can provide the plurality of population response profiles to the learning algorithm 210.

At block 1514, the controller 200 receives additional kinematic data, satisfaction data, and therapy parameters for at least one patient. At least some of the data can be extracted features taken from the collected signals. In some embodiments, the controller 200 collects data measured during a therapy session. In some embodiments, the controller 200 collects data measured at times outside the therapy session.

At block 1516, the controller 200 aggregates the additional data. At block 1518, the controller 200 provides the additional aggregated data to the aggregation learning algorithm 208. The aggregation learning algorithm 208 can determine parameters of one or more response profiles from the additional aggregated data set. The aggregation learning algorithm 208 can use the additional aggregated features collected by the aggregation algorithm 206 to update the one or more response profiles that correspond to one or more different neurostimulation therapies. The aggregation learning algorithm 208 can be associated with one or more of the neurostimulation device 100, the user interface device 150, the base station 120, and/or the cloud 122.

At block 1520, the controller 200 receives a plurality of updated response profiles from the aggregation learning algorithm 208. At block 1522, the controller 200 can provide the updated plurality of population response profiles to the learning algorithm 210.

The process can return to block 1514 where the controller 200 can receive additional kinematic data, satisfaction data, and/or therapy parameters for the therapy session. The process can then repeat blocks 1514-1522 to further iterate the therapy parameters to improve outcomes of the patient.

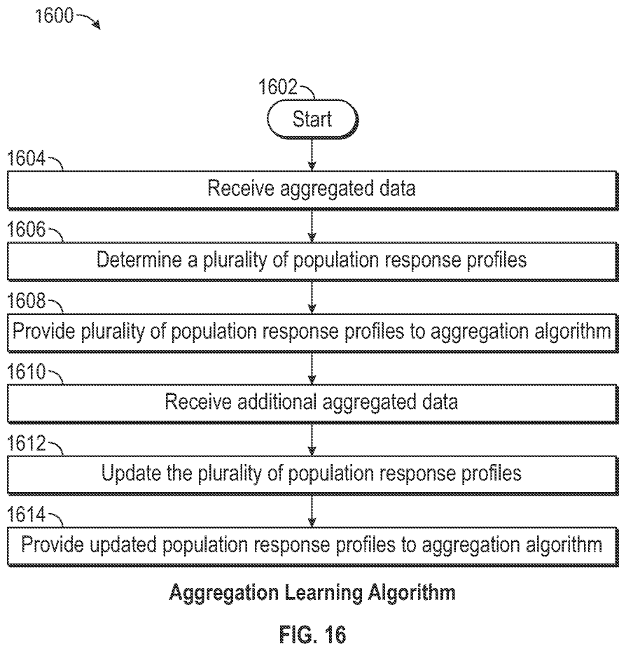

FIG. 16 illustrates a flow chart of a non-limiting embodiment of a process 1600 for personalizing therapeutic protocols employing the aggregated data from FIG. 15. The process 1600 can be implemented by any of the systems described above.

The process begins at block 1602 and then moves to block 1604 where the controller 200 receives the data aggregated in block 1508 of FIG. 15. In some embodiments, the aggregation learning algorithm 208 receives the aggregated data.

At block 1606, the aggregation learning algorithm 208 can determine parameters of one or more response profiles from the aggregated data set. In some embodiments, the aggregation learning algorithm 208 determines a plurality of population response profiles. The aggregation learning algorithm 208 can evaluate the extracted features (e.g., for both treatment metrics and/or kinematic metrics) based on geospatial data, temporal data, and device characteristics associated with the extracted features. The aggregation learning algorithm 208 can use the aggregated features within the aggregated data to determine the one or more response profiles that correspond to one or more different neurostimulation therapies. The aggregation learning algorithm 208 can be associated with one or more of the neurostimulation device 100, the user interface device 150, the base station 120, and/or the cloud 122.

At block 1608, the aggregation learning algorithm 208 can provide the plurality of population response profiles to the aggregation algorithm 206 at block 1510 of FIG. 15. At block 1610 the controller 200 receives additional aggregated data aggregated by the aggregation algorithm 206 at block 1518 of FIG. 15.

At block 1612, the aggregation learning algorithm 208 can update the plurality of population response profiles. The aggregation learning algorithm 208 can use the additional aggregated features collected by the aggregation algorithm 206 to update the one or more response profiles that correspond to one or more different neurostimulation therapies. The aggregation learning algorithm 208 can be associated with one or more of the neurostimulation device 100, the user interface device 150, the base station 120, and/or the cloud 122.

At block 1614, the aggregation learning algorithm 208 provides the updated population response profiles to the aggregation algorithm 206. The process can return to block 1610 where the aggregation learning algorithm 208 receives additional aggregated data aggregated by the aggregation algorithm 206 at block 1518 of FIG. 15.

FIG. 17 is a table of sample parameters of response profiles that can be employed by the systems disclosed herein.

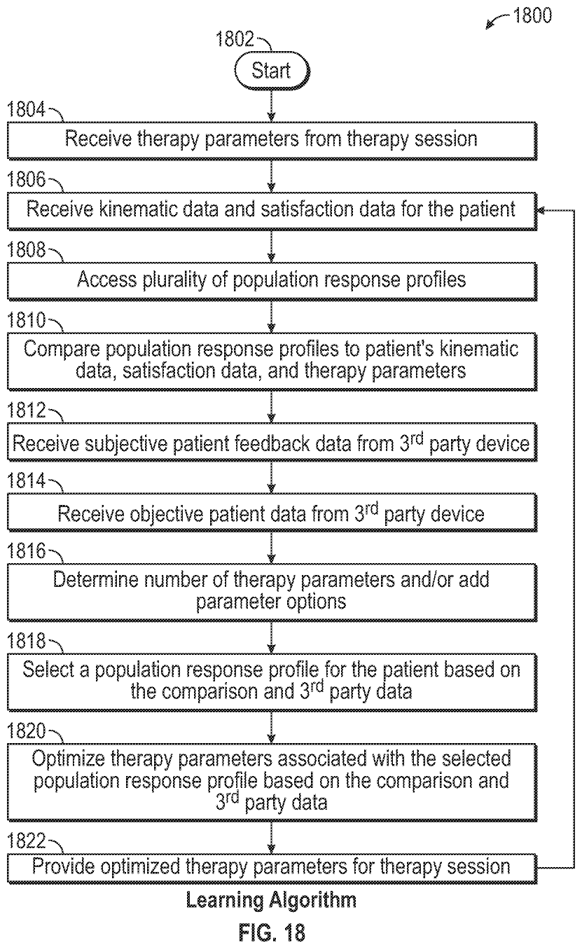

FIG. 18 illustrates a flow chart of a non-limiting embodiment of a machine learning process 1800 employed by the methods disclosed in any of FIGS. 12-15. The process 1800 can be implemented by any of the systems described above.

The process begins at block 1802 and then moves to block 1804 where the learning algorithm 210 receives the therapy parameters from the therapy session. In some embodiments, therapy parameters are listed in FIG. 17. At block 1806, In some embodiments, the learning algorithm 210 receives kinematic data and satisfaction data from the patient. In some embodiments, data can include:
Pre-therapy-session kinematics features (Frequency power features: Peak frequency in tremor band (4-12 hz), Amplitude at Peak frequency, Power Spectral Density (PSD) integral in frequency window around peak, Q-factor-narrow, Q-factor-wide, Spectral Entropy, PSD integral of tremor band, PSD integral ratio of narrow in wide frequency band, PSD integral of low frequency band (0-4 hz));
Times-series feature (Approximate entropy, cycle peak-to-peak amplitude, cycle peak-to-peak amplitude (mean), cycle peak-to-peak amplitude (std), cycle peak-to-peak duration (mean), cycle peak-to-peak duration (std));
Session-specific timing features (Session local start time: Hour of the day, Day of the week, am or PM, Part of day (in three-hour segments), Lapsed time to previous session, Session Number within current day, Number of sessions since the first session, Days since the first session, Actively used days since the first session); and
Session-specific physical measurements (AC Impedance, BC impedance).

At block 1808, the learning algorithm 210 accesses the plurality of population responses profiles created by the aggregation learning algorithm 208. At block 1810, the learning algorithm 210 compares the population response profiles to the patient's kinematic data, satisfaction data, and therapy parameters.

At block 1812, In some embodiments, the learning algorithm 210 receives subjective patient feedback data from third party devices. The third party devices can include the user interface device 150. At block 1814, In some embodiments, the learning algorithm 210 receives objective patient feedback data from third party devices.

At block 1816, the learning algorithm 210 determines the number of therapy parameters and/or adds parameter options. At block 1818, the learning algorithm 210 can select a population response profile for the patient based on the comparison with the population response profiles and the third party data.

At block 1820, In some embodiments, the learning algorithm 210 optimizes the therapy parameters associated with the selected population response profile for the patient based on the comparison with the population response profiles and the third party data. As explained below, the inputs to the learning algorithm 210 at blocks 1816 and/or 1820 can further include:
Demographics/Static features (Age, Sex, Race, Ethnicity, BMI, Wrist band size, wrist circumference, hand of treatment, hand-dominant, ET-onset age, ET-responsive to alcohol, ET-family history, ET-diagnosis age, Medication-prior ET, Medication-Botox, Medication-current ET, Medication-antidepressants, ET-diagnosis age difference from onset age, Years since diagnosis, Years since onset, Quality of life satisfaction (QUEST). Clinicians-rated scores prior to treatment, Mental health features).

At block 1822, the learning algorithm 210 can provide the optimized therapy parameters for the therapy session. In some embodiments, the learning algorithm 210 provides the optimized therapy parameters to the neurostimulation device 100.

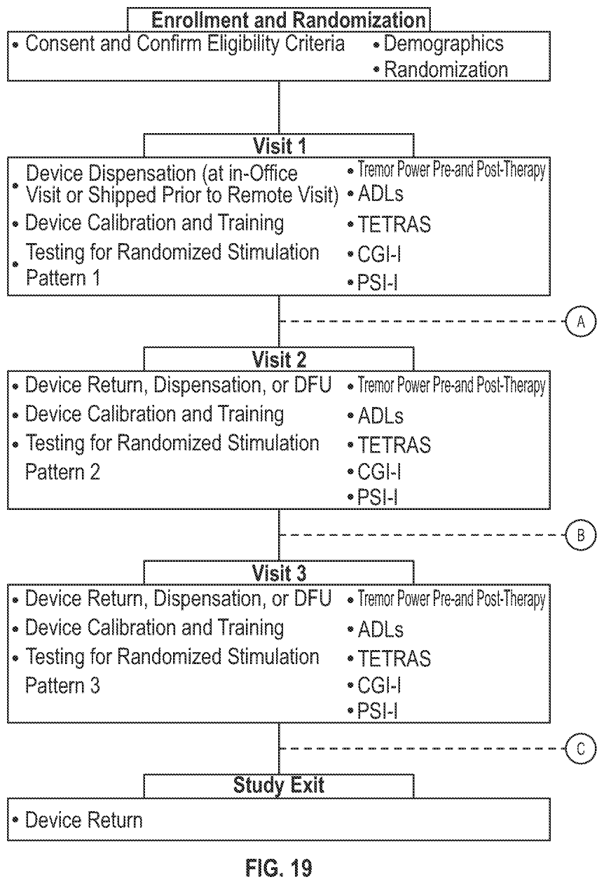

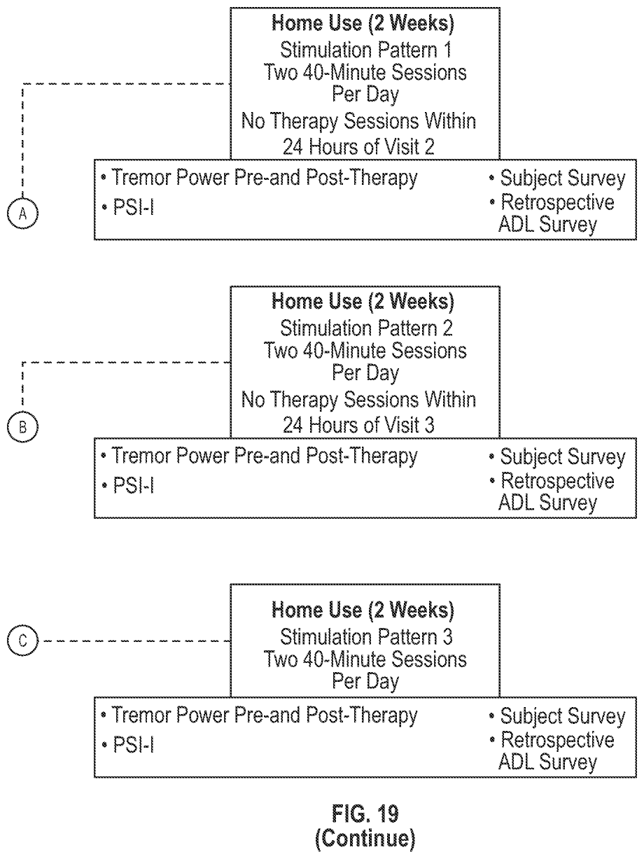

FIG. 19 illustrates an overall patient study used to develop treatment algorithms disclosed herein. The patient study was a randomized clinical study that including having the patients experience three different types of therapy waveforms. The data from this study demonstrates the advantage of employing predictive capability to determine a best of three waveforms for a specific patient. For example, In some embodiments, the learning algorithm 210 employs machine learning to predict kinematic improvement and/or patient ratings. The predicted kinematic improvement and/or patient ratings can be used by the learning algorithm 210 to recommend an improved therapy. The improved therapy can include identify the best waveform for the specific patient. A specific example, method, and evaluation schematic are described herein.

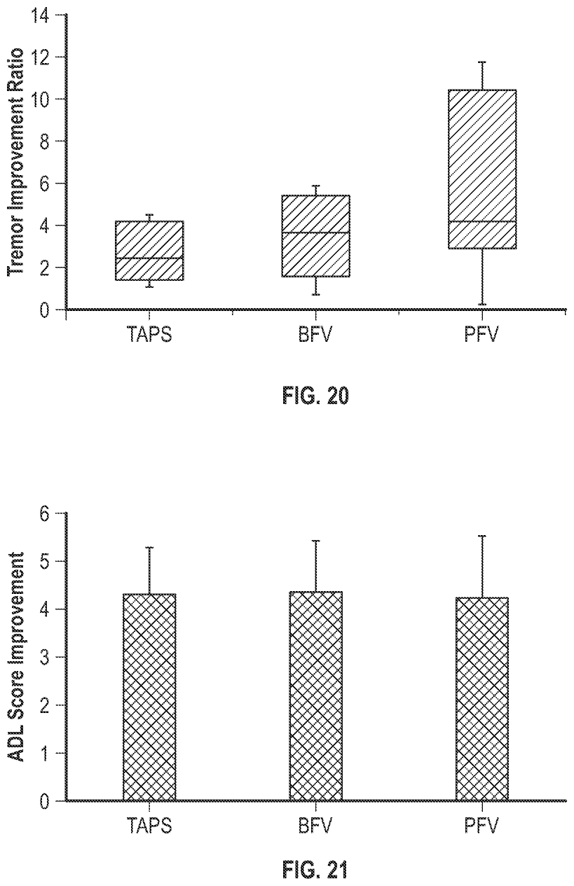

FIG. 20 is a graph of patient tremor improvement ratios from the study for different waveforms based on measured kinematic data of the patient. As shown by the data, the waveforms included transcutaneous afferent patterned stimulation (TAPS), burst frequency jitter (BFV), and pulse frequency jitter (PFV). The primary kinematic endpoint as illustrated in FIG. 20 is a tremor improvement ratio. The tremor improvement ratio was a ratio of pre-stimulation or pre-therapy tremor power to post-stimulation or post-therapy tremor power. For reference an improvement ratio of 2 is the same as a 50% reduction in tremor power. From the study, we determined about half of the patients had a 2-fold or greater improvement. An improvement ratio<1 indicates a worsening or increase in tremor power.

FIG. 21 is a bar chart of patient activities of daily living (ADL) scores for different waveforms based on subjective data from the patients in the study. The secondary endpoint as illustrated in FIG. 21 is a patient-rated ADL. For reference the ADL's included eight different tasks that were rated by the patient. An ADL score improvement of 4 equates to a 1 point improvement in about four of the eight rated tasks.

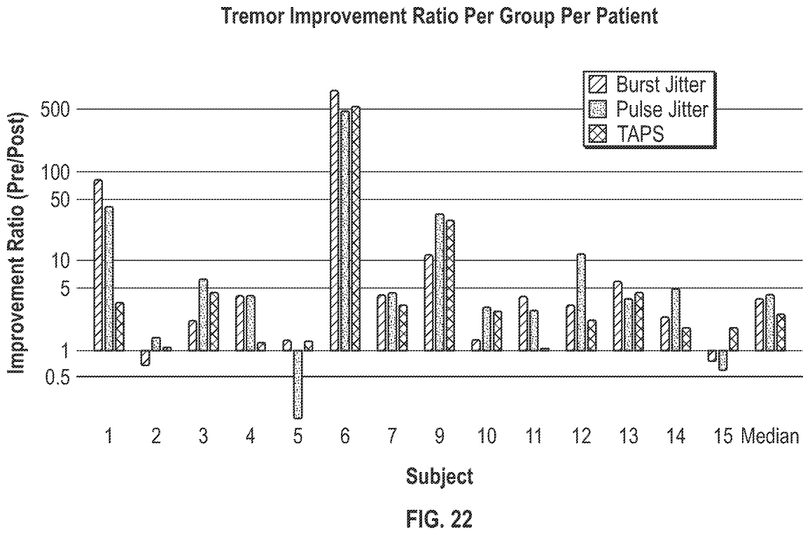

FIG. 22 is a bar chart of patient tremor improvement ratios for different waveforms based on measured kinematic data for individual patients in the study. As illustrated in FIG. 22, patients responded differently to each of the waveforms (TAPS, pulse frequency jitter, and burst frequency jitter).

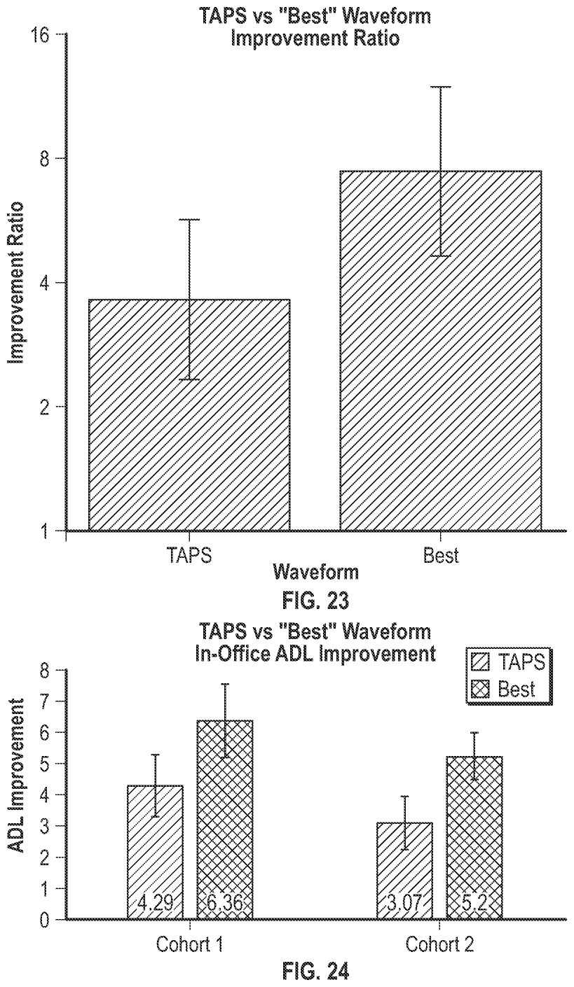

FIG. 23 is a bar chart of potential patient tremor improvement ratios for TAPS vs. the best waveform from the study. FIG. 23 shows motion sensor-rated improvements for all of the patients' TAPS (left) waveform vs. the “best” waveform (right) for all of the patients.

FIG. 24 includes bar charts of potential ADL scores for TAPS and the best waveform for both cohorts 1 and 2. FIG. 24 shows patient-rated improvements for all of the patients' TAPS (left) vs. the “best” waveform (right). We learned the rankings did not translate across the metrics in that the “best” sensor-rated waveforms differed from the “best” patient-rated waveforms.

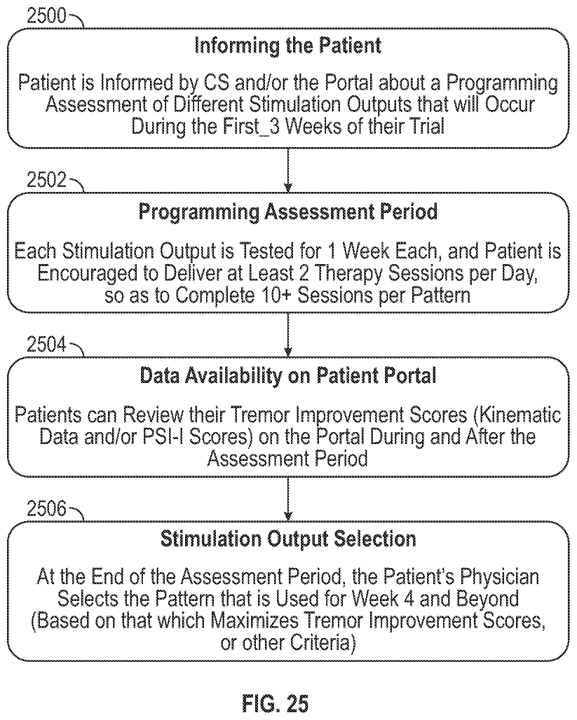

FIG. 25 illustrates an embodiment of a method for selecting a stimulation output. The method begins at block 2500 where the patient is informed about a programming assessment of different stimulation outputs that will occur during a trial. The illustrated trial is 3 weeks. In some embodiments, during week one the output is fixed (e.g., TAPS). During week two the output is a Dynamic Burst Output or burst frequency jitter. During week three the output is Dynamic Pulse Output or pulse frequency jitter.

The method moves to block 2502 for a programming assessment period. During the programming assessment period, each stimulation output waveform is tested for 1 week each, and the patient is encouraged to deliver at least 2 therapy sessions per day so as to complete at least 10 sessions per waveform pattern. The method moves to block 2504 where the patient is able to review their tremor improvement scores (kinematic data and/or PSI-I scores) on a user portal during and after the assessment period. The method then moves to block 2506 where the stimulation output is selected. At the end of the assessment period, In some embodiments, the patient's physician selects the waveform pattern that is used for Week 4 and beyond. The waveform can be selected based on which waveform maximizes tremor improvement scores or other criteria.

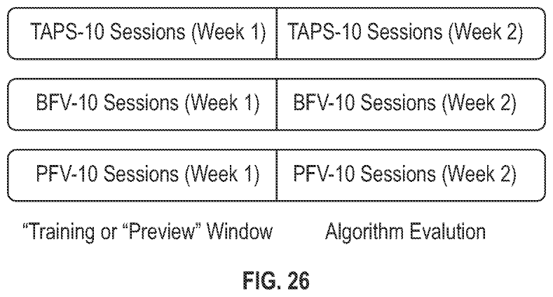

FIG. 26 illustrates a proposed timing for a method that includes a training or preview window and an algorithm evaluation period.

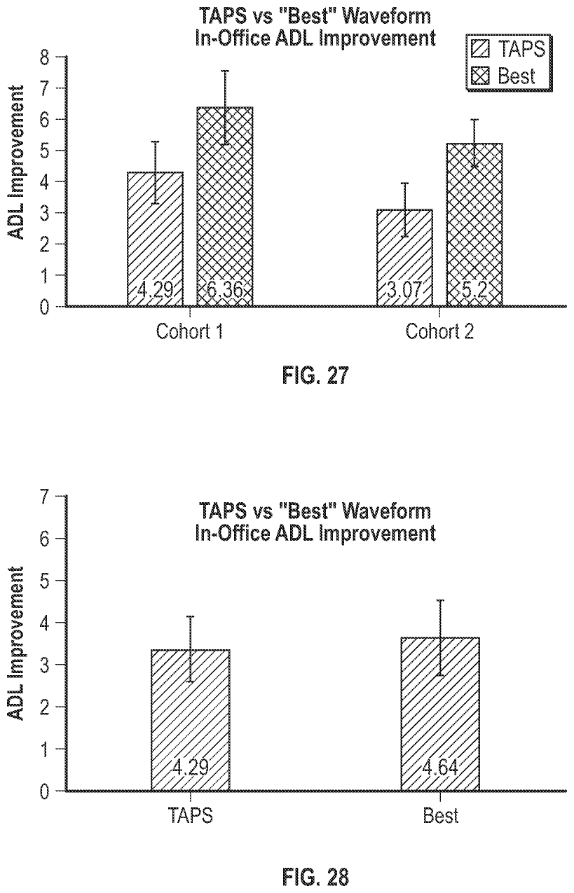

FIG. 27 illustrates the ADL scores for TAPS and the best waveform for both cohorts 1 and 2 that are associated with the best ADL scores. FIG. 28 illustrates the ADL scores for TAPS and the best waveform for cohort 1 that is associated with the best kinematic data. As is shown by FIGS. 27 and 28, the best waveform identified by the ADL rankings for cohort 1 provides a greater improvement in ADL score than the best waveform identified for cohort 1 based on kinematic ranking. In this way, many of the methods and systems disclosed herein rely at least in part on patient satisfaction scores.

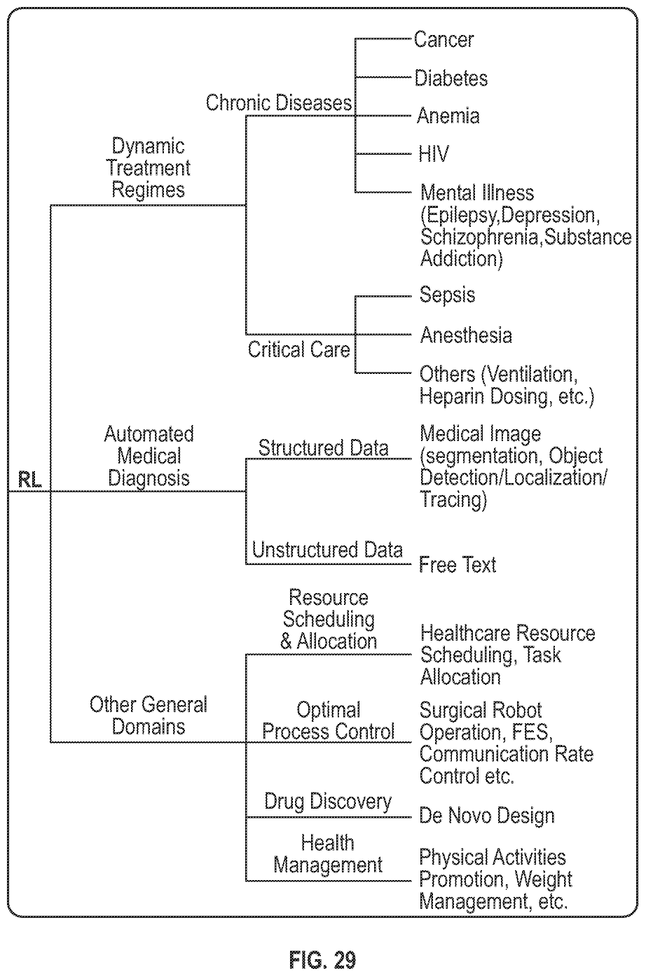

FIG. 29 illustrates healthcare domains where machine learning, including reinforcement learning (RL), disclosed herein can be implemented. Machine learning including RL can be applied in a number of healthcare domains, which are broadly organized into FIG. 29. Thus, this disclosure is not limited to any particular field. Reinforcement learning (RL) is a subfield in machine learning and can be applied to real-life problems.

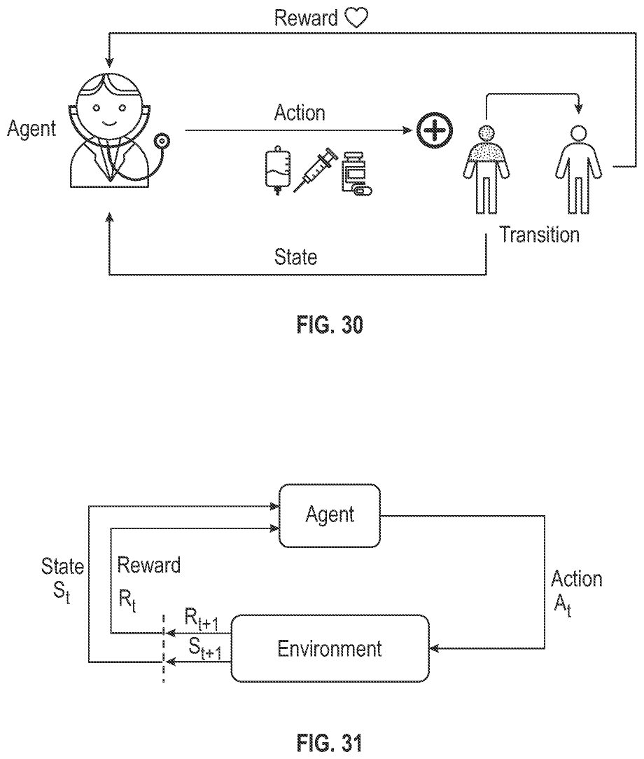

FIG. 30 schematically illustrates a reinforcement learning system represented by an agent which can be employed in the systems disclosed herein. As an example, In some embodiments, RL can employ an agent which chooses an action at each time step based on its current state. In some embodiments, the agent receives an evaluative feedback and the new state from the environment. The goal of the agent can be to learn an optimal policy (mapping from the states to actions) that maximizes the accumulated reward it receives over time. In this way, the agent does not receive direct instructions regarding which actions they should take. Instead, the agent learns which actions are the best through trial-and-error interactions with the environment.

This adaptive closed loop feature renders RL distinct from traditional supervised learning methods for regression in which a list of correct labels must be provided. In some embodiments, RL does not require a well-represented, first-principle derived mathematical model of the environment, nor an explicitly defined decision-tree. Instead, RL develops a control policy directly from experience. This can make RL more appealing since it could be difficult to build an accurate model for the human body and the response to administered treatment, due to non-linear, varying, and delayed interactions between treatment and human bodies.

Typically, a medical treatment regime is composed of a sequence of decisions to determine the course of decisions such as treatment type, drug dosage, or re-examination timing, according to the current health status and prior treatment history of an individual patient, with a goal of promoting the patient's long-term benefits. RL can be tailored for achieving precise treatment for individual patients who may possess high heterogeneity in response to the treatment.

In some embodiments, the learning algorithm 210 performs RL. Of course, the learning algorithm 210 is not limited to performing RL. In some embodiments that employ RL, RL is used to optimize therapy outcome. In some embodiments, the methods disclosed herein focus on continuous state-space modeling, finding suitable actions and learned treatment policies that could improve patient outcomes.

In some embodiments, reinforcement learning system can employ a state representation. For example, in certain embodiment, a time stamp “t” can be defined as a single therapy session, or a group of six consecutive sessions with state features aggregated to ensure stability. In some embodiments, at every time step t, the agent observes the current state of the environment St. In some embodiments, choices of features that can represent a current state include Demographics/Static (Age, Sex, Race, Ethnicity, BMI, Wrist band size, wrist circumference, hand of treatment, hand-dominant, ET-onset age, ET-responsive to alcohol, ET-family history, ET-diagnosis age, Medication-prior ET, Medication-Botox, Medication-current ET, Medication-antidepressants, ET-diagnosis age difference from onset age, Years since diagnosis, Years since onset, Quality of life satisfaction (QUEST). Clinicians-rated scores prior to treatment, Mental health features); Pre-therapy-session kinematics features (Frequency power features: Peak frequency in tremor band (4-12 hz), Amplitude at Peak frequency, Power Spectral Density (PSD) integral in frequency window around peak, Q-factor-narrow, Q-factor-wide, Spectral Entropy, PSD integral of tremor band, PSD integral ratio of narrow in wide frequency band, PSD integral of low frequency band (0-4 hz)); Times-series features (Approximate entropy, cycle peak-to-peak amplitude, cycle peak-to-peak amplitude (mean), cycle peak-to-peak amplitude (std), cycle peak-to-peak duration (mean), cycle peak-to-peak duration (std)); Session-specific timing features (Session local start time (Hour of the day, Day of the week, am or PM, Part of day (in three-hour segments), Lapsed time to previous session, Session Number within current day, Number of sessions since the first session, Days since the first session, Actively used days since the first session)); and Session-specific physical measurements (AC Impedance, BC impedance). In some embodiments, the included features can be considered to represent important parameters clinicians would examine when deciding treatment and dosage for patients.

### Action and Rewards

In some embodiments, once the state St is observed, the agent takes an action at. In some embodiments, a 4×2×2×2×3 action space was defined for the firmware interventions considering the state space St upon completion of pre-therapy measurements. Continuing with this example, the action space defines 96 unique combinations of parameters values in total.
Therapy type: 40 min, 2 hour, 8×10, kick start
Jitter: Yes, No
Anatomical Jitter: Yes, No
Amplitude: dependent, independent
Amplitude level: perception, comfortable, maximum

The action space can be represented as tuples, e.g., (40 min, Jitter, No Anatomical Jitter, Independent Amplitude, Comfortable Amplitude setting). In some embodiments, the agent selects actions that maximize its expected discounted future reward, defined as

$$\text{?} ( s ) \hat{=} \text{?} [ {\sum }_{l = 0}^{∞} {γ}^{t} ℛ ( \text{?} , π ( {s}_{t} ) ) \vert {s}_{0} = s ]$$

$$\text{?} \text{indicates text missing or illegible when filed}$$

where the discount factor γ accounts for immediate reward being more valuable than future rewards and the reward function r (t′)=C₀(patient rated improvement at timestamp t)+C₁(pre/post accelerator measured fold improvement at timestamp t). In some embodiments, multiple weight values for C₀ and C₁ were defined based on correlation between patient rated improvement and kinematic improvement. The policy IT maps states to actions, ensuring maximum discounted expected reward after executing action a in state s, expressed as

$$Q * ( s , a ) = {max}_{π} 𝔼 [ {R}_{t} \vert {s}_{t} = s , {a}_{t} = a , π ] .$$

FIG. 31 illustrates a reinforcement learning algorithm performed by the agent of FIG. 30. In the description below, the elements of a Markov decision process (MDP) are used as a general framework to formalize an RL problem=(S, A, P, R, γ). S is defined as the state space consisting of St. A is the set of actions available to the agent with at denoting the action that the agent programs at time t, P(s, a, s′). S×A×S->[0, 1] is a Markovian transition function when the agent transits from state s to state s′ after taking action a. R: S×A>R is a reward function that returns the immediate rewards R(s, a) to the agent after taking action a in state s. γ∈[0, 1] is a discount factor.

The MDP problem computes an optimal policy. In some embodiments, the MDL problem can employ model-based or model-free methods. For example, whether to employ model-based or model-free methods can depend on whether a complete knowledge of the MDP model is specified. In some embodiments, the focus can be on model-free methods such as learning methods. Learning methods learn an optimal policy simply based on received observations and rewards.

There can be at least two main model-free control techniques, Q-learning and SARSA (State-action-reward-state-action). Q-learning can be advantageous when the goal is to train an optimal agent in simulation or in a low-cost and fast-iterating environment. Q-learning has a direct learning optimal policy. SARSA can be advantageous when the agent learns online, and our goal is for rewards to be gained while learning. In embodiments where there are limited timestamps and to ensure maximal treatment success for patients, SARSA may be employed instead of Q-learning. In some embodiments, pseudocode of a SARSA implementation is shown below.

| | |
|---|---|
| 1. | Initialize t = 0. |
| 2. | Start with S₀ and choose action A₀ = arg ma [symbol] Q(S₀, a), where ∈-greedy is commonly |
| | applied. |
| 3. | At time t, after applying action Aₜ, we observe reward Rₜ₊₁ and get into the next state Sₜ₊₁. |
| 4. | Then pick the next action in the same way as in step 2: Aₜ₊₁ = arg m [symbol] Q(Sₜ₊₁, a). |
| 5. | Update the Q-value function: |
| | Q(Sₜ, Aₜ) ← Q(Sₜ, Aₜ) + a(Rₜ₊₁ + γQ(Sₜ₊₁, Aₜ₊₁) − Q(Sₜ, Aₜ)). |
| 6. | Set t = t + 1 and repeat from step 3. |

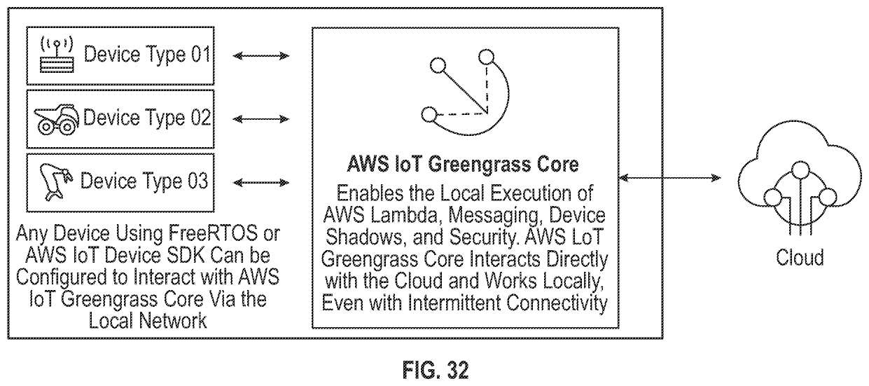

FIG. 32 illustrates an engineering implementation of the machine learning algorithm of FIGS. 30 and 31. In the engineering implementation, setting up the RL process and delivering models to edge devices at patients' homes and on the go is done in the cloud 122. In the engineering implementation, the base station 120 hosts cloud computing services (e.g., AWS IoT Greengrass Devices) that run software (e.g., Linux such as Ubuntu and Raspbian and support ARM or x86 architectures). The base station 120 can be connected to the cloud 122 or internet via any wired or wireless connection protocol. The base station 120 can comprise a computation device (e.g., Intel Atom, NVIDIA Jetson TX2, and Raspberry Pi). In certain implementations, the cloud computing services use the models built, and trained in the cloud and run inference locally on devices, with pre-built runtimes for common ML frameworks. The base station 120 generates actions and commands which are sent securely to the neurostimulation device 100.

In addition to model inference, the base station 120 can perform computationally intensive tasks including transient movement signal removal, time series feature compute that would otherwise be infeasible to compute on the neurostimulation device 100. A base station 120 that has a computationally capable but economically scalable computation device can future-proof the need for computation by the base station 120.

In some embodiments, the neurostimulation device 100 can include pre-built connectors and configured to communicate with the base station 120 over a local network (i.e. low-power Bluetooth or wired connection). The neurostimulation device 100 executes the command and actions receive from the base station 120. Microcontroller firmware residing in the neurostimulation device 100 is set up to execute actions to set stimulation parameters near-real-time.

### Terminology

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps. However, some embodiments can consist or consist essentially of any number of stated elements or steps disclosed herein.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The methods disclosed herein include certain actions taken by a practitioner; however, they can also include any third-party instruction of those actions, either expressly or by implication. For example, actions such as “percutaneously stimulating an afferent peripheral nerve” includes “instructing the stimulation of an afferent peripheral nerve.”

1.-120. (canceled)

121. A wearable neurostimulation device for transcutaneously stimulating one or more peripheral nerves of a user, the device comprising:
one or more electrodes configured to generate electric stimulation signals based on therapy parameters, the therapy parameters being selected from a plurality of predefined profiles determined by an aggregation learning algorithm, the aggregation learning algorithm predicting a plurality of outcomes for the user based on a plurality of predefined profiles, the plurality of predefined profiles being based on features extracted from data for a plurality of users;
one or more sensors configured to detect physiological data, wherein the one or more sensors are operably connected to the wearable neurostimulation device; and
one or more hardware processors configured to:
perform a therapy session with the therapy parameters;
measure physiological data from the one or more sensors during the therapy session, wherein the physiological data includes one or more of heart rate, blood glucose, blood pressure, respiration rate, body temperature, blood volume, sound pressure, photoplethysmography, electroencephalogram, electrocardiogram, blood oxygen saturation, and/or skin conductance data;
adjust the therapy parameters based on the measured physiological data from the therapy session; and
optimize the adjusted therapy parameters by a learning algorithm based on an individual database, the learning algorithm predicting a modified outcome based on the individual database which includes an aggregation of physiological data and satisfaction data from multiple therapy sessions for the user.

122. The device of claim 121, wherein the physiological data includes respiration rate and heart rate, and wherein the one or more hardware processors is configured to treat depression.

123. The device of claim 121, wherein the one or more sensors are further configured to detect sleep patterns and activity level of the user.

124. The device of claim 121, wherein the one or more hardware processors is configured to treat migraine or Lyme disease.

125. A neurostimulation device for stimulating one or more peripheral nerves of a user, the device comprising:
one or more electrodes configured to generate electric stimulation signals based on therapy parameters;
one or more sensors configured to detect physiological data, wherein the one or more sensors are operably connected to the neurostimulation device; and
one or more hardware processors configured to:
receive a set of therapy parameters;
perform a therapy session with the set of therapy parameters;
collect physiological and user satisfaction data for the therapy session, wherein the physiological data includes one or more of heart rate, blood glucose, blood pressure, respiration rate, body temperature, blood volume, sound pressure, photoplethysmography, electroencephalogram, electrocardiogram, blood oxygen saturation, and/or skin conductance data;
provide the set of therapy parameters, the physiological data, and the user satisfaction data to a learning algorithm;
receive optimized therapy parameters from the learning algorithm, the learning algorithm predicting a modified outcome based on an individual database which includes an aggregation of physiological data and satisfaction data from multiple therapy sessions for the user; and
perform another therapy session with the optimized therapy parameters, wherein the device is a wearable transcutaneous device.

126. The device of claim 125, wherein the physiological data includes respiration rate and heart rate, and wherein the one or more hardware processors is configured to treat depression.

127. The device of claim 125, wherein the one or more sensors are further configured to detect sleep patterns and activity level of the user.

128. The device of claim 125, wherein the one or more hardware processors is configured to treat migraine or Lyme disease.

129. A wearable neurostimulation device for transcutaneously stimulating one or more peripheral nerves of a user, the device comprising:
one or more electrodes configured to generate electric stimulation signals based on therapy parameters, the therapy parameters being selected from a plurality of predefined profiles determined by an aggregation learning algorithm, the aggregation learning algorithm predicting a plurality of outcomes for the user based on a plurality of predefined profiles, the predefined profiles being based on features extracted from data for a plurality of users;
one or more sensors configured to detect motion signals, wherein the one or more sensors are operably connected to the wearable neurostimulation device; and
one or more hardware processors configured to:
perform a therapy session with the therapy parameters;
measure kinematic data including data from the detected motion signals during the therapy session, wherein the data includes tremor data;
adjust the therapy parameters based on measured data from the detected motion signals during the therapy session; and
optimize the adjusted therapy parameters by a learning algorithm based on an individual database, the learning algorithm predicting a modified outcome based on the individual database which includes an aggregation of kinematic data and satisfaction data from multiple therapy sessions for the user.

130. The wearable neurostimulation device according to claim 129, wherein the features extracted from the data include test kinematic data.

131. The wearable neurostimulation device according to claim 129, wherein the features extracted from the data include satisfaction data.

132. The wearable neurostimulation device according to claim 129, wherein the therapy parameters are based at least in part on a predetermined decision tree.

133. The wearable neurostimulation device according to claim 129, wherein the therapy parameters are based at least in part on one or more user profiles.

134. The wearable neurostimulation device according to claim 129, wherein the kinematic data is collected from a sensor onboard the neurostimulation device.

135. The wearable neurostimulation device according to claim 129, wherein the kinematic data includes at least one of accelerometer data and gyroscope data.

136. The wearable neurostimulation device according to claim 129, wherein the features extracted from the data for the plurality of users include one or more of geospatial data, temporal data, disease, patient attributes or characteristics, or neurostimulation device characteristics associated with the extracted features.

137. The wearable neurostimulation device according to claim 129, wherein the individual database includes at least one of: (i) kinematic data accumulated over multiple days and (ii) satisfaction data accumulated over multiple days.

138. The wearable neurostimulation device according to claim 129, wherein the device is configured to be worn on at least one of: a wrist, a leg, and an ear.

139. The wearable neurostimulation device of claim 129, wherein the one or more peripheral nerves includes at least one of a median, radial, ulnar, sural, femoral, peroneal, saphenous, or tibial nerve.

140. The wearable neurostimulation device of claim 129, wherein the device includes a stimulator and detachable band, the stimulator supporting the one or more hardware processors and the detachable band supporting the one or more electrodes.
